(19) United States
(12) Patent Application Publication
(10) Pub. No.: US 2024/0226308 A1
(43) Pub. Date: Jul. 11, 2024

(54) DOSING REGIMENS OF PEPTIDE CONJUGATES OF TOPOISOMERASE I INHIBITORS

(71) Applicant: CYBREXA 2, INC., New Haven, CT (US)

(72) Inventors: Vishwas PARALKAR, New Haven, CT (US); Arthur P. DECILLIS, New Haven, CT (US)

(21) Appl. No.: 18/288,650

(22) PCT Filed: Apr. 28, 2022
(86) PCT No.: PCT/US2022/071967

§ 371 Date: Oct. 27, 2023

Related U.S. Application Data
(60) Provisional application No. 63/181,640, filed on Apr. 29, 2021.

(51) Int. Cl. A61K 47/64; A61K 9/00; A61P 35/00

(52) U.S. Cl. CPC A61K 47/64; A61K 9/0019; A61P 35/00

(57) ABSTRACT

The present invention relates to dosing regimens of peptide conjugates of toporsomcrase I inhibitors (e.g., a peptide conjugate of the topoisomerase I inhibitor exatecan), which is useful for the treatment of diseases such as cancer.

### FIELD OF THE INVENTION

The present invention relates to dosing regimens of peptide conjugates of topoisomerase I inhibitors (e.g., a peptide conjugate of the topoisomerase I inhibitor exatecan), which is useful for the treatment of diseases such as cancer.

### SEQUENCE LISTING

This application contains a Sequence Listing that has been submitted electronically as an ASCII text file named 43236-0019WO1_ST25.txt. The ASCII text file, created on Apr. 26, 2022, is 2.20 kilobytes in size. The material in the ASCII text file is hereby incorporated by reference in its entirety.

### BACKGROUND OF THE INVENTION

Cancer is a group of diseases characterized by aberrant control of cell growth. The annual incidence of cancer is estimated to be in excess of 1.6 million in the United States alone. While surgery, radiation, chemotherapy, and hormones are used to treat cancer, it remains the second leading cause of death in the U.S. It is estimated that about 600,000 Americans will die from cancer each year.

Treatment of cancer in humans by systemic administration of pharmaceutical agents often functions by slowing or terminating the uncontrolled replication that is a characteristic of cancer cells. One class of such agents is topoisomerase I inhibitors. Topoisomerase 1 enzymes function to relax supercoiled DNA and alleviate DNA helical constraints and play a role in transcriptional regulation. See Li, M., Genomics Proteomics Bioinformatics 14 (2016), 166-171. Topoisomerase I is essential for the development in the mammalian system due to its dynamic functions in DNA replication and transcription. However, due to its direct role in transcriptional regulation, topoisomerase I dysfunction may lead to abnormal cellular functions. See Li, M., Genomics Proteomics Bioinformatics 14 (2016). 166-171. Thus. several human diseases such as cancer, neurodegenerative diseases, and autoimmune diseases, are linked to topoisomerase I regulation and activity.

Inhibitors of topoisomerase I have been developed and continue to be developed as anti-cancer agents. In particular, topoisomerase I inhibitors are widely used for the treatment of colorectal, gastric, and other cancers. See Ogitani, Bioorg. Med. Chem. Lett. 26 (2016), 5069-5072. Although topoisomerase I inhibitors are useful in the treatment of cancer, the compounds also exhibit side effects, including neutropenia and severe diarrhea. Preferential delivery of topoisomerase inhibitors to these diseased tissues could avoid these serious side effects. Peptide conjugates of topoisomerase I inhibitors are described in US Patent Application Publication No. US 2021/009719. There is a need for the further development of peptide conjugates of topoisomerase inhibitors, and dosing regimens thereof.

### SUMMARY

The present disclosure provides, inter alia, a method of treating cancer in a patient, comprising administering to said patient a compound of Formula (I):

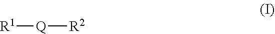

or a pharmaceutically acceptable salt thereof, wherein:
R¹ is a peptide;
R² is a small molecule topoisomerase I targeting moiety, which binds to topoisomerase I; and
Q is a linker, which is covalently linked to moiety R¹ and R²;
wherein the compound, or the pharmaceutically acceptable salt thereof, is administered in a daily dose of about 0.1 mg/kg to about 1.5 mg/kg as measured by the amount of the free form of the compound.

The present disclosure provides, inter alia, a method of treating cancer in a patient, comprising administering to said patient Compound 1 having the structure:

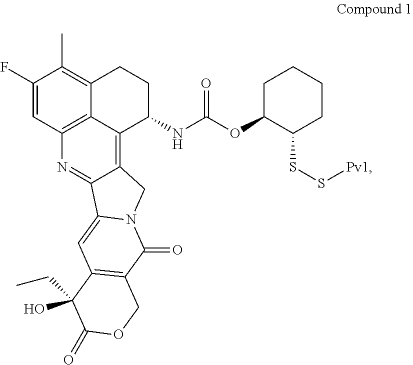

or a pharmaceutically acceptable salt thereof, wherein Pv1 is a peptide comprising the following sequence:
ADDQNPWRAYLDLLFPTDTLLLDLLWCG (SEQ ID NO: 1); and
   wherein Compound 1 or the pharmaceutically acceptable salt thereof is administered in a daily dose of about 0.1 mg/kg to about 1.5 mg/kg as measured by the amount of the free form of Compound 1.

The present disclosure further provides a method of treating cancer in a patient, comprising administering to said patient a compound of Formula (I):

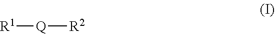

or a pharmaceutically acceptable salt thereof, wherein:
R¹ is a peptide;
R² is a small molecule topoisomerase I targeting moiety, which binds to topoisomerase 1; and
Q is a linker, which is covalently linked to moiety R¹ and R²;
wherein the compound, or the pharmaceutically acceptable salt thereof is administered in a daily dose of about 5 mg/m² to about 100 mg/m² as measured by the amount of the free form of the compound.

The present disclosure further provides a method of treating cancer in a patient, comprising administering to said patient Compound 1 having the structure:

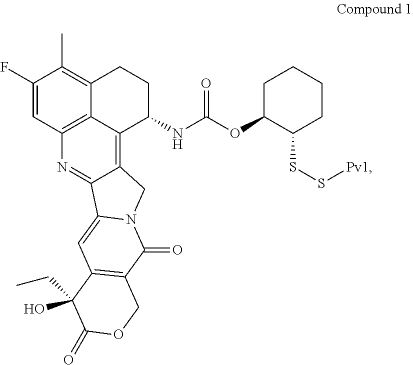

or a pharmaceutically acceptable salt thereof, wherein Pv1 is a peptide comprising the following sequence:
ADDQNPWRAYLDLLFPTDTLLLDLLWCG (SEQ ID NO: 1); and
   wherein Compound 1 or the pharmaceutically acceptable salt thereof is administered in a daily dose of about 5 mg/m² to about 100 mg/m² as measured by the amount of the free form of Compound 1.

The present disclosure further provides a method of administering Compound 1, or a pharmaceutically acceptable salt thereof, to a patient in need of treatment, comprising parenterally delivering to said patient Compound 1 having the structure:

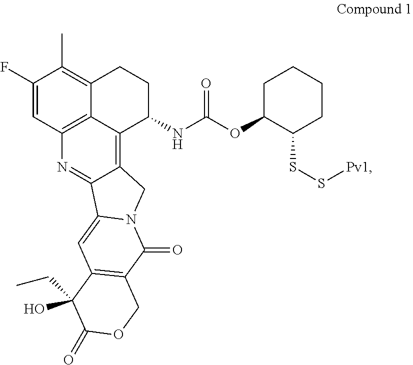

or a pharmaceutically acceptable salt thereof, wherein Pv1 is a peptide comprising the following sequence:
ADDQNPWRAYLDLLFPTDTLLLDLLWCG (SEQ ID NO: 1); and
   wherein Compound 1 or the pharmaceutically acceptable salt thereof is administered in a daily dose of about 0.1 mg/kg to about 1.5 mg/kg as measured by the amount of the free form of Compound 1.

The present disclosure further provides a method of administering Compound 1, or a pharmaceutically acceptable salt thereof, to a patient in need of treatment, comprising parenterally delivering to said patient Compound 1 having the structure:

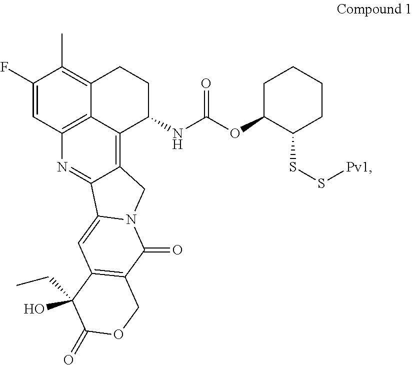

or a pharmaceutically acceptable salt thereof, wherein Pv1 is a peptide comprising the following sequence:
ADDQNPWRAYLDLLFPTDTLLLDLLWCG (SEQ ID NO: 1); and
   wherein Compound 1 or the pharmaceutically acceptable salt thereof is administered in a daily dose of about 5 mg/m² to about 100 mg/m² as measured by the amount of the free form of Compound 1.

The present disclosure further provides a use of a compound of Formula (1) or a pharmaceutically acceptable salt thereof in the preparation of a medicament for the treatment of cancer.

The present disclosure further provides a compound of Formula (1) or a pharmaceutically acceptable salt thereof for use in any of the methods described herein.

The present disclosure further provides a use of Compound 1 or a pharmaceutically acceptable salt thereof in the preparation of a medicament for the treatment of cancer.

The present disclosure further provides Compound 1 or a pharmaceutically acceptable salt thereof for use in any of the methods described herein.

### BRIEF DESCRIPTION OF THE DRAWINGS

FIG. 1A shows the mean plasma levels of Compound 1 and exatecan (Cohort A) at 1 day.

FIG. 1B shows the mean plasma levels of Compound 1 and exatecan (Cohort A) at 4 days.

FIG. 2A shows the mean plasma levels of Compound 1 and exatecan (Cohort B) at 1 day.

FIG. 2B shows the mean plasma levels of Compound 1 and exatecan (Cohort B) at 3 days.

### DETAILED DESCRIPTION

### Administration of a Compound of Formula (I)

The present disclosure provides, inter alia, a method of treating cancer in a patient, comprising administering to said patient a compound of Formula (I):

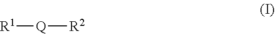

or a pharmaceutically acceptable salt thereof, wherein:
R¹ is a peptide;
R² is a small molecule topoisomerase I targeting moiety, which binds to topoisomerase I; and
Q is a linker, which is covalently linked to moicty R¹ and R^{2;}
   wherein the compound, or the pharmaceutically acceptable salt thereof, is administered in a daily dose of about 0.1 mg/kg to about 1.5 mg/kg as measured by the amount of the free form of the compound.

The present disclosure further provides a method of treating cancer in a patient, comprising administering to said patient a compound of Formula (1):

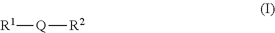

or a pharmaceutically acceptable salt thereof, wherein:
R¹ is a peptide;
R² is a small molecule topoisomerase I targeting moiety, which binds to topoisomerase I;
Q is a linker, which is covalently linked to moiety R¹ and R²; and
   wherein the compound, or the pharmaceutically acceptable salt thereof, is administered in a daily dose of about 5 mg/m² to about 100 mg/m² as measured by the amount of the free form of the compound.

The present disclosure further provides a method of administering a compound of Formula (1):

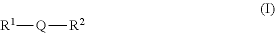

or a pharmaceutically acceptable salt thereof, to a patient in need of treatment, comprising parenterally delivering the compound to said patient, wherein:
R¹ is a peptide;
R² is a small molecule topoisomerase I targeting moiety, which binds to topoisomerase I; and
Q is a linker, which is covalently linked to moicty R¹ and R²;
wherein the compound, or the pharmaceutically acceptable salt thereof, is administered in a daily dose of about 0.1 mg/kg to about 1.5 mg/kg as measured by the amount of the free form of the compound.

The present disclosure further provides a method of administering a compound of Formula (1):

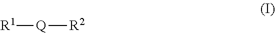

or a pharmaceutically acceptable salt thereof, to a patient in need of treatment, comprising parenterally delivering the compound to said patient, wherein:
R¹ is a peptide;
R² is a small molecule topoisomerase I targeting moiety, which binds to topoisomerase I; and
Q is a linker, which is covalently linked to moiety R¹ and R²;
wherein the compound, or the pharmaceutically acceptable salt thereof, is administered in a daily dose of about 5 mg/m² to about 100 mg/m² as measured by the amount of the free form of the compound

The present disclosure further provides a method of administering a compound of Formula (I):

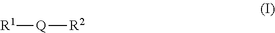

or a pharmaceutically acceptable salt thereof, to a patient in need of treatment, comprising parenterally delivering to said patient the compound, or a pharmaceutically acceptable salt thereof, wherein:
R¹ is a peptide;
R¹ is a small molecule topoisomerase I targeting moiety, which binds to topoisomerase I; and
Q is a linker, which is covalently linked to moicty R¹ and R²;
wherein the compound, or the pharmaceutically acceptable salt thereof, is administered in a daily dose of about 5 mg/m² to about 100 mg/m² as measured by the amount of the free form of the compound.

The present disclosure further provides a method of administering a compound of Formula (I):

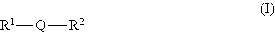

or a pharmaceutically acceptable salt thereof, to a patient in need of treatment, comprising parenterally delivering to said patient the compound, or a pharmaceutically acceptable salt thereof, wherein:
R¹ is a peptide:
R² is a small molecule topoisomerase I targeting moiety, which binds to topoisomerase 1; and
Q is a linker, which is covalently linked to moiety R¹ and R²;
wherein the compound, or the pharmaceutically acceptable salt thereof, is administered in a daily dose of about 5 mg/m² to about 100 mg/m² as measured by the amount of the free form of the compound.

In some embodiments, the peptide of R¹ bas 10 to 50 amino acids. 20 to 40 amino acids, 10 to 20 amino acids, 20 to 30 amino acids, or 30 to 40 amino acids.

In some embodiments, the peptide of R¹ is a conformationally restricted peptide. A conformationally restricted peptide can include, for example, macrocyclic peptides and stapled peptides. A stapled peptide is a peptide constrained by a covalent linkage between two amino acid side-chains, forming a peptide macrocycle. Conformationally restricted peptides are described, for example, in Guerlavais et al., Annual Reports in Medicinal Chemistry 2014, 49, 331-345; Chang et al., Proceedings of the National Academy of Sciences of the United States of America (2013), 110(36), E3445-E3454; Tesauro et al., Molecules 2019, 24, 351-377; Dougherty et al., Journal of Medicinal Chemistry (2019), 62(22), 10098-10107; and Dougherty et al., Chemical Reviews (2019). 119(17), 10241-10287, cach of which is incorporated herein by reference in its entirety.

In some embodiments, the peptide of R¹ is an environmentally sensitive peptide described, for example, in U.S. Pat. Nos. 8,076,451 and 9,289,508 and U.S. Pat. Pub. No. 2019/209580 (each of which are incorporated herein by reference in their entirety), although other peptides capable of such selective insertion could be used. Other suitable peptides are described, for example, in Wecrakkody, et al., PNAS 110 (15), 5834-5839 (Apr. 9, 2013), which is also incorporated herein by reference in its entirety. Without being bound by theory, it is believed that the environmentally sensitive peptide undergoes a conformational change and inserts across cell membranes in response to physiological changes (e.g., pH). The peptide can target acidic tissue and selectively translocate polar, cell-impermeable molecules across cell membranes in response to low extracellular pH. In some embodiments, the peptide is capable of selectively delivering molecules across a cell membrane having an acidic or hypoxic mantle having a pH less than about 6.0. In some embodiments, the peptide is capable of selectively delivering a molecule across a cell membrane having an acidic or hypoxic mantle having a pH less than about 6.5. In some embodiments, the peptide is capable of selectively delivering a molecule across a cell membrane having an acidic or hypoxic mantle having a pH less than about 5.5. In some embodiments, the peptide is capable of selectively delivering a molecule across a cell membrane having an acidic or hypoxic mantle having a pH between about 5.0 and about 6.0.

The term “acidic and/or hypoxic mantle” refers to the environment of the cell in the diseased tissue in question having a pH lower than 7.0 and preferably lower than 6.5. An acidic or hypoxic mantle more preferably has a pH of about 5.5 and most preferably has a pH of about 5.0. The compounds of formula (I) insert across a cell membrane having an acidic and/or hypoxic mantle in a pH dependent fashion to insert R²- into the cell. whereupon the disulfide linker is cleaved to deliver free R²H. Since the compounds of formula (I) are pH-dependent, they preferentially insert across a cell membrane only in the presence of an acidic or hypoxic mantle surrounding the cell and not across the cell membrane of “normal” cells, which do not have an acidic or hypoxie mantle. An example of a cell having an acidic or hypoxic mantle is a cancer cell.

The terms “pH-sensitive” or “pH-dependent” as used herein to refer to the peptide R¹ or to the mode of insertion of the peptide RI or of the compounds of the invention across a cell membrane, means that the peptide has a higher affinity to a cell membrane lipid bilayer having an acidic or hypoxic mantle than a membrane lipid bilayer at neutral pH. Thus, the compounds of the invention preferentially insert through the cell membrane to insert R²- to the interior of the cell (and thus deliver R²H as described above) when the cell membrane lipid bilayer has an acidic or hypoxic mantle (a “diseased” cell) but does not insert through a cell membrane when the mantle (the environment of the cell membrane lipid bilayer) is not acidic or hypoxic (a “normal” cell). It is believed that this preferential insertion is achieved as a result of the peptide R¹ forming a helical configuration, which facilitates membrane insertion.

In some embodiments, the peptide of R¹ comprises at least one of the following sequences:

(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG;
(SEQ ID NO. 3; Pv3)
ADDQNPWRAYLDLLFPTDTLLLDLLWDADECG;
(SEQ ID NO. 4; Pv4)
Ac-AAEQNPIYWARYADWLFTTPLLLLDLALLVDADEGTKCG;
and
(SEQ ID No. 5; Pv5)
AAEQNPIYWARYADWLFTTPLLLLDLALLVDADEGTC.

In some embodiments, the peptide of R¹ comprises at least one of the following sequences:

(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG.

In some embodiments, the peptide of R¹ comprises the sequence

(SEQ ID NO. 1; Pv1)
ADDQNPWRAYLDLLFPTDTLLLDLLWCG.

In some embodiments, the peptide of R¹ comprises the sequence

(SEQ ID NO. 2; Pv2)
AEQNPIYWARYADWLFTTPLLLLDLALLVDADECG.

In some embodiments, the peptide of R¹ comprises the sequence

(SEQ ID NO. 3; Pv3)
ADDQNPWRAYLDLLFPTDTLLLDLLWDADECG.

In some embodiments, the peptide of R¹ comprises the sequence

(SEQ ID NO. 4; Pv4)
Ac-AAEQNPIYWARYADWLFTTPLLLLDLALLVDADEGTKCG.

In some embodiments, the peptide of R¹ comprises the sequence

(SEQ ID NO. 5; Pv5)
AAEQNPIYWARYADWLFTTPLLLLDLALLVDADEGTC.

In some embodiments, the peptide of R¹ consists essentially of the sequence

(SEQ ID NO. 1; Pv1)
ADDQNPWRAYLDLLFPTDTLLLDLLWCG.

In some embodiments, the peptide of R¹ consists essentially of the sequence

(SEQ ID NO. 2; Pv2)
AEQNPIYWARYADWLFTTPLLLLDLALLVDADECG.

In some embodiments, the peptide of R¹ consists essentially of the sequence

(SEQ ID NO. 3; Pv3)
ADDQNPWRAYLDLLFPTDTLLLDLLWDADECG.

In some embodiments, the peptide of R¹ consists essentially of the sequence

(SEQ ID NO. 4; Pv4)
AAEQNPIYWARYADWLFTTPLLLLDLALLVDADEGTKCG.

In some embodiments, the peptide of R¹ consists essentially of the sequence

(SEQ ID NO. 5; Pv5)
AAEQNPIYWARYADWLFTTPLLLLDLALLVDADEGTC.

Additional peptides are disclosed in in U.S. Patent Publication No. US 2019/209580, U.S. patent application Ser. No. 16/925,094, and U.S. patent application Ser. No. 16/924,445, each of which is incorporated herein in its entirety.

The term “small molecule topoisomerase I targeting moiety” or “topoisomerase I inhibitor” refers to a chemical group that binds to topoisomerase I. The small molecule topoisomerase I targeting moiety can be a group derived from a compound that inhibits the activity of topoisomerase I. Topoisomerase inhibitors include camptothecin and derivatives and analogues thereof such as opotecan, irinotecan (CPT-11), silatecan (DB-67, AR-67), cositecan (BNP-1350), lurtotecan, gimatecan (ST1481), belotecan (CKD-602), rubitecan, topotecan, deruxtecan, and exatecan. Topoisomerase inhibitors are described in, for example, Ogitani, Bioorg. Med. Chem. Lett. 26 (2016), 5069-5072; Kumazawa, E., Cancer Chemother Pharmacol 1998, 42: 210-220; Tahara, M, Mol Cancer Ther 2014, 13(5): 1170-1180; Nakada, T., Bioorganic & Medicinal Chemistry Letters 2016, 26: 1542-1545.

Compounds of Formula (I) having a topoisomerase I targeting moiety are described in U.S. Patent Application Publication No. 2021/0009719. In some embodiments of compounds of Formula (I), R¹ is camptothecin, opotecan, irinotecan (CPT-11), silatecan (DB-67, AR-67), cositecan (BNP-1350), lurtotecan, gimatecan (ST1481), belotecan (CKD-602), rubitecan, topotecan, deruxtecan, or exatecan. In some embodiments of compounds of Formula (I), R² is exatecan.

The moiety Q is a linking group, covalently connecting R¹ and R² that serves a tether between the peptide and topoisomerase I inhibitor that may be cleaved when the conjugate or portion thereof is inside a cell. In some embodiments. Q is a chain of 1 to 40, 1 to 30, 1 to 25. 1 to 20, 1 to 15, 1 to 10, or 1 to 5 chain atoms, which is optionally substituted with 1-10 R^{q} substituents, and wherein one or more chain carbon atoms of Q can be oxidized to form a carbonyl (C═O), and wherein one or more N and S chain atoms can each be optionally oxidized to form an amine oxide, sulfoxide or sulfonyl group; wherein
each R^{q} is independently selected from OH, CN, —COOH, NH₂, balo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl. C₃₋₆ cycloalkyl, NH(C₁₋₆ alkyl) and N(C₁₋₆ alkyl)₂, wherein the C₁₋₆ alkyl, phenyl, C₃₋₆ cycloalkyl, 4-6 membered beterocycloalkyl, and 5-6 membered heteroaryl of R″ are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C3-10 cycloalkyl, 5- or 6-membered heteroaryl or 4-6 membered heterocycloalkyl; and
two Rº groups together with the chain atoms to which they are attached can form a phenyl, 5-6 membered heteroaryl, 4-6 membered heterocycloalkyl, or C₃₋₆ cycloalkyl ring.

In some embodiments, Rª is independently selected from OH, CN, —COOH, NH₂, balo, C₁₋₆ haloalkyl, C₁₋₆ alkyl, C₁₋₆ alkoxy, C₁₋₆ haloalkoxy, NH(C₁₋₆ alkyl) and N(C₁₋₆ alkyl)₂.

In some embodiments, the compound is a compound of Formula (II):

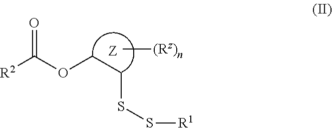

or a pharmaceutically acceptable salt thereof, wherein:
R¹ is a peptide:
R² is a topoisomerase I inhibitor;

Ring Z is a monocyclic C₅₋₇ cycloalkyl ring or a monocyclic 5-7 membered beterocycloalkyl ring:
each R^{Z} is independently selected from C₁₋₄ alkyl, halo, CN, NO₂, OR^{a1}, SR^{a1}, C(O)R^{b1}, C(O)NR^{c1}IR^{d1}, C(O)OR^{a1}, OC(O)R^{b1}, OC(O)NR^{c1}R^{d1}, NR^{c1}R^{d1}, NRC^{c1}C(O)R^{b1}, NR^{c1}(O)OR^{a1}, and NR^{c1}C(O)NR^{c1}R^{d1};
or two adjacent R^{Z} together with the atoms to which they are attached form a fused monocyclic C₅₋₇ cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NRC^{c1}C(O)R^{b1}, NR^{c1}(O)OR^{a1}, and NR^{c1}C(O)NR^{c1}R^{d1};
R^{a1}, R^{b1}, R^{c1}, and R^{d1} are cach independently selected from H, C₁₋₄ alkyl, C₂₋₄ alkenyl, C₂₋₄ alkynyl, cach optionally substituted with 1, 2, or 3 substituents independently selected from halo, OH, CN, and NO₂; and
n is 0, 1, 2, or 3.

In some embodiments of compounds of Formula (II), R¹ is a peptide comprising the sequence of SEQ ID NO: 1, SEQ ID NO:2, SEQ ID NO:3, SEQ ID NO:4, or SEQ ID NO:5.

In some embodiments of compounds of Formula (II), R¹ is Pv1, Pv2, Pv3, Pv4, or Pv5.

In some embodiments of compounds of Formula (II), R¹ is attached to the core via a cysteine residue of R¹ wherein one of the sulfur atoms of the disulfide moiety in Formula II is derived from the cysteine residue.

In some embodiments of compounds of Formula (II), R¹ is camptothecin, opotecan, irinotecan (CPT-11), silatecan (DB-67, AR-67), cositecan (BNP-1350), lurtotecan, gimatecan (ST1481), belotecan (CKD-602), rubitecan, topotecan, deruxtecan, or exatecan.

In some embodiments of compounds of Formula (II), R² is exatecan.

In some embodiments of compounds of Formula (II), R² is attached to the core through an N atom.

In some embodiments of compounds of Formula (II), Ring Z is a monocyclic C₅₋₇ cycloalkyl ring.

In some embodiments of compounds of Formula (II), Ring Z is a cyclopentyl ring.

In some embodiments of compounds of Formula (II), Ring Z is a cyclohexyl ring.

In some embodiments of compounds of Formula (II), Ring Z is a cycloheptyl ring.

In some embodiments of compounds of Formula (II), Ring Z is a monocyclic 5-7 membered heterocycloalkyl ring.

In some embodiments of compounds of Formula (II), Ring Z is a 5-membered heterocycloalkyl ring.

In some embodiments of compounds of Formula (II), Ring Z is a 6-membered heterocycloalkyl ring.

In some embodiments of compounds of Formula (II), Ring Z is a 7-membered heterocycloalkyl ring.

In some embodiments of compounds of Formula (II), two adjacent RZ together with the atoms to which they are attached form a fused monocyclic C₅₋₇cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NRC^{c1}C(O)R^{b1}, NR^{c1}(O)OR^{a1}, and NR^{c1}C(O)NR^{c1}R^{d1}.

In some embodiments of compounds of Formula (II), n is 0.

In some embodiments of compounds of Formula (II), n is 1.

In some embodiments of compounds of Formula (II), n is 2.

In some embodiments of compounds of Formula (II), n is 3.

In some embodiments, the compounds of the invention is a compound of Formula (III), Formula (IV), or Formula (V):

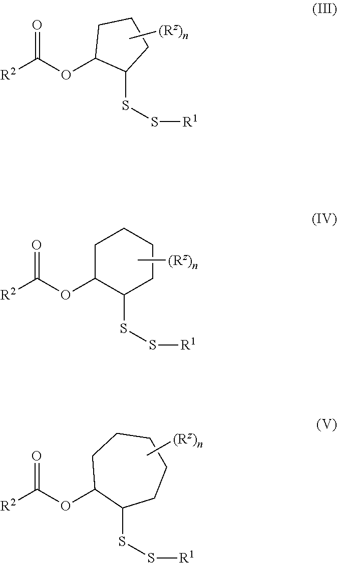

or a pharmaceutically acceptable salt thereof, wherein R¹, R², RZ and n are defined as in any of the embodiments above for Formula (II).

In some embodiments, the compound is Compound 1, having the structure:

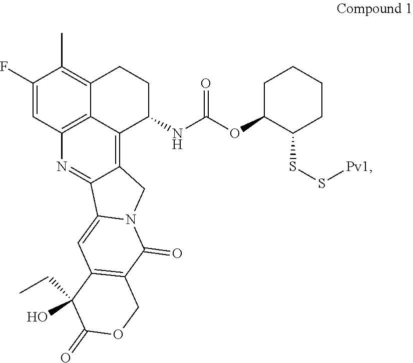

or a pharmaceutically acceptable salt thereof, wherein Pv1is a peptide comprising the following sequence:

(SEQ ID NO: 1)
ADDQNPWRAYLDLLFPTDTLLLDLLWCG.

In some embodiments, the method comprises administering to said patient a compound of Formula (I), or a pharmaceutically acceptable salt thereof, wherein the compound, or the pharmaceutically acceptable salt thereof, is administered in a daily dose of about 0.1 mg/kg to about 1.5 mg/kg as measured by the amount of the free form of the compound. In some embodiments, the compound, or a pharmaceutically acceptable salt thereof, is administered in a daily dose of about 0.25 mg/kg to about 1.5 mg/kg as measured by the amount of the free form of the compound. In some embodiments, the compound, or a pharmaceutically acceptable salt thereof, is administered in a daily dose of about 0.1 mg/kg to 1.25 mg/kg as measured by the amount of the free form of the compound. In some embodiments, the compound, or a pharmaceutically acceptable salt thereof, is administered in a daily dose of about 0.25 mg/kg to about 1.25 mg/kg as measured by the amount of the free form of the compound. In some embodiments, the compound, or a pharmaceutically acceptable salt thereof, is administered in a daily dose of about 0.25 mg/kg to about 0.75 mg/kg as measured by the amount of the free form of the compound. In some embodiments, the compound, or a pharmaceutically acceptable salt thereof, is administered in a daily dose of about 0.25 mg/kg to about 0.50 mg/kg as measured by the amount of the free form of the compound. In some embodiments, the compound, or a pharmaceutically acceptable salt thereof, is administered in a daily dose of about 0.5 mg/kg to about 0.75 mg/kg as measured by the amount of the free form of the compound. In some embodiments. the compound, or a pharmaceutically acceptable salt thereof, is administered in a daily dose of about 0.5 mg/kg to about 1.25 mg/kg as measured by the amount of the free form of the compound.

Provided herein is a method of treating cancer in a patient, comprising administering to said patient a compound of Formula (I), wherein the compound is administered in a daily dose of about 0.1 mg/kg to about 1.5 mg/kg. In some embodiments. the compound is administered in a daily dose of about 0.25 mg/kg to about 1.5 mg/kg. In some embodiments, the compound is administered in a daily dose of about 0.1 mg/kg to 1.25 mg/kg. In some embodiments. the compound is administered in a daily dose of about 0 25 mg/kg to about 1.25 mg/kg. In some embodiments, the compound is administered in a daily dose of about 0.25 mg/kg to about 0.75 mg/kg. In some embodiments, the compound is administered in a daily dose of about 0.25 mg/kg to about 0.50 mg/kg. In some embodiments. the compound is administered in a daily dose of about 0.5 mg/kg to about 0.75 mg/kg. In some embodiments, the compound is administered in a daily dose of about 0.5 mg/kg to about 1.25 mg/kg.

In some embodiments, the compound, or a pharmaceutically acceptable salt thereof, is administered in a daily dose of about 0.25 mg/kg as measured by the amount of the free form of the compound. In some embodiments. the compound, or a pharmaceutically acceptable salt thereof, is administered in a daily dose of about 0.5 mg/kg as measured by the amount of the free form of the compound. In some embodiments, the compound, or a pharmaceutically acceptable salt thereof, is administered in a daily dose of about 0.75 mg/kg as measured by the amount of the free form of the compound. In some embodiments, the compound, or a pharmaceutically acceptable salt thereof, is administered in a daily dose of about 1.0 mg/kg as measured by the amount of the free form of the compound. In some embodiments. the compound, or a pharmaceutically acceptable salt thereof, is administered in a daily dose of about 1.25 mg/kg as measured by the amount of the free form of the compound.

In some embodiments, the compound is administered in a daily dose of about 0.25 mg/kg. In some embodiments, the compound is administered in a daily dose of about 0.5 mg/kg. In some embodiments, the compound is administered in a daily dose of about 0.75 mg/kg. In some embodiments, the compound is administered in a daily dose of about 1.0 mg/kg. In some embodiments, the compound is administered in a daily dose of about 1.25 mg/kg.

In some embodiments, the compound, or a pharmaceutically acceptable salt thereof, is administered in a daily dose of about 1.5 mg/kg or less as measured by the amount of the free form of the compound. In some embodiments, the compound, or a pharmaceutically acceptable salt thereof, is administered in a daily dose of about 1.25 mg/kg or less as measured by the amount of the free form of the compound. In some embodiments, the compound, or a pharmaceutically acceptable salt thereof, is administered in a daily dose of about 1.0 mg/kg or less as measured by the amount of the free form of the compound. In some embodiments, the compound, or a pharmaceutically acceptable salt thereof, is administered in a daily dose of about 0.75 mg/kg or less as measured by the amount of the free form of the compound. In some embodiments, the compound, or a pharmaceutically acceptable salt thereof, is administered in a daily dose of about 0.5 mg/kg or less as measured by the amount of the free form of the compound. In some embodiments, the compound, or a pharmaceutically acceptable salt thereof, is administered in a daily dose of about 0.25 mg/kg or less as measured by the amount of the free form of the compound.

In some embodiments, the compound is administered in a daily dose of about 1.5 mg/kg or less. In some embodiments, the compound is administered in a daily dose of about 1.25 mg/kg or less. In some embodiments, the compound is administered in a daily dose of about 1.0 mg/kg or less. In some embodiments, the compound is administered in a daily dose of about 0.75 mg/kg or less. In some embodiments, the compound is administered in a daily dose of about 0.5 mg/kg or less. In some embodiments, the compound is administered in a daily dose of about 0.25 mg/kg or less.

In some embodiments, the compound, or a pharmaceutically acceptable salt thereof, is administered intravenously. In some embodiments. the compound, or a pharmaceutically acceptable salt thereof, is administered as an intravenous infusion. In some embodiments, the compound, or a pharmaceutically acceptable salt thereof, is administered as an intravenous infusion in a dosage of about 0.1 mg/kg to about 1.5 mg/kg as measured by the amount of the free form of the compound. In some embodiments, the compound, or a pharmaceutically acceptable salt thereof, is administered as an intravenous infusion in a dosage of about 0.25 mg/kg to about 1.5 mg/kg as measured by the amount of the free form of the compound. In some embodiments, The compound, or a pharmaceutically acceptable salt thereof, is administered as an intravenous infusion in a dosage of about 0.1 mg/kg to about 1.25 mg/kg as measured by the amount of the free form of the compound. In some embodiments. the compound, or a pharmaceutically acceptable salt thereof, is administered as an intravenous infusion in a dosage of about 0.25 mg/kg to about 1.25 mg/kg as measured by the amount of the free form of the compound. In some embodiments, the compound, or a pharmaceutically acceptable salt thereof, is administered as an intravenous infusion in a dosage of about 0.25 mg/kg to about 0.75 mg/kg as measured by the amount of the free form of the compound. In some embodiments. the compound, or a pharmaceutically acceptable salt thereof, is administered as an intravenous infusion in a dosage of about 0.25 mg/kg to about 0.5 mg/kg as measured by the amount of the free form of the compound. In some embodiments, the compound, or a pharmaceutically acceptable salt thereof, is administered as an intravenous infusion in a dosage of about 0.5 mg/kg to about 0.75 mg/kg as measured by the amount of the free form of the compound. In some embodiments. the compound, or a pharmaceutically acceptable salt thereof, is administered as an intravenous infusion in a dosage of about 0.5 mg/kg to about 1.25 mg/kg as measured by the amount of the free form of the compound. In some embodiments. the compound, or a pharmaceutically acceptable salt thereof, is administered as an intravenous infusion in a dosage of about 0.5 mg/kg to about 1.5 mg/kg as measured by the amount of the free form of the compound. In some embodiments, the compound, or a pharmaceutically acceptable salt thereof, is administered as an intravenous infusion in a dosage of about 0.25 mg/kg as measured by the amount of the free form of the compound. In some embodiments, the compound, or a pharmaceutically acceptable salt thereof, is administered as an intravenous infusion in a dosage of about 0.5 mg/kg as measured by the amount of the free form of the compound. In some embodiments, the compound, or a pharmaceutically acceptable salt thereof, is administered as an intravenous infusion in a dosage of about 0.75 mg/kg as measured by the amount of the free form of the compound. In some embodiments, the compound, or a pharmaceutically acceptable salt thereof, is administered as an intravenous infusion in a dosage of about 1.0 mg/kg as measured by the amount of the free form of the compound. In some embodiments, the compound, or a pharmaceutically acceptable salt thereof, is administered as an intravenous infusion in a dosage of about 1.25 mg/kg as measured by the amount of the free form of the compound.

In some embodiments, the compound is administered as an intravenous infusion in a dosage of about 0.1 mg/kg to about 1.5 mg/kg. In some embodiments, the compound is administered as an intravenous infusion in a dosage of about 0.25 mg/kg to about 1.5 mg/kg. In some embodiments, the compound is administered as an intravenous infusion in a dosage of about 0.1 mg/kg to about 1.25 mg/kg. In some embodiments. the compound is administered as an intravenous infusion in a dosage of about 0.25 mg/kg to about 1.25 mg/kg. In some embodiments, the compound is administered as an intravenous infusion in a dosage of about 0.25 mg/kg to about 0.75 mg/kg. In some embodiments, the compound is administered as an intravenous infusion in a dosage of about 0.25 mg/kg to about 0.5 mg/kg. In some embodiments, the compound is administered as an intravenous infusion in a dosage of about 0.5 mg/kg to about 0.75 mg/kg. In some embodiments, the compound is administered as an intravenous infusion in a dosage of about 0.5 mg/kg to about 1.25 mg/kg. In some embodiments, the compound is administered as an intravenous infusion in a dosage of about 0.5 mg/kg to about 1.5 mg/kg. In some embodiments, the compound is administered as an intravenous infusion in a dosage of about 0.25 mg/kg. In some embodiments, the compound is administered as an intravenous infusion in a dosage of about 0.5 mg/kg. In some embodiments, the compound is administered as an intravenous infusion in a dosage of about 0.75 mg/kg. In some embodiments, the compound is administered as an intravenous infusion in a dosage of about 1.0 mg/kg. In some embodiments. the compound is administered as an intravenous infusion in a dosage of about 1.25 mg/kg.

Provided herein is a method of treating cancer in a patient, comprising administering to said patient a compound of Formula (I), or a pharmaceutically acceptable salt thereof. wherein the compound, or the pharmaceutically acceptable salt thereof, is administered in a daily dose of about 5 mg/m² to about 100 mg/m² as measured by the amount of the free form of the compound. In some embodiments, the compound, or a pharmaceutically acceptable salt thereof, is administered in a daily dose of about 10 mg/m² to about 100 mg/m² as measured by the amount of the free form of the compound. In some embodiments, the compound, or a pharmaceutically acceptable salt thereof, is administered in a daily dose of about 5 mg/m² to about 80 mg/m² as measured by the amount of the free form of the compound. In some embodiments, the compound, or a pharmaceutically acceptable salt thereof, is administered in a daily dose of about 10 mg/m² to about 80 mg/m² as measured by the amount of the free form of the compound. In some embodiments, the compound, or a pharmaceutically acceptable salt thereof, is administered in a daily dose of about 20 mg/m² to about 60 mg/m² as measured by the amount of the free form of the compound. In some embodiments, the compound, or a pharmaceutically acceptable salt thereof, is administered in a daily dose of about 20 mg/m² to about 100 mg/m² as measured by the amount of the free form of the compound. In some embodiments, the compound, or a pharmaceutically acceptable salt thereof, is administered in a daily dose of about 20 mg/m² to about 45 mg/m² as measured by the amount of the free form of the compound. In some embodiments, the compound, or a pharmaceutically acceptable salt thereof, is administered in a daily dose of about 20 mg/m² to about 30 mg/m² as measured by the amount of the free form of the compound. In some embodiments, the compound, or a pharmaceutically acceptable salt thereof, is administered in a daily dose of about 10 mg/m² to about 60 mg/m² as measured by the amount of the free form of the compound. In some embodiments, the compound, or a pharmaceutically acceptable salt thereof, is administered in a daily dose of about 10 mg/m² to about 45 mg/m² as measured by the amount of the free form of the compound. In some embodiments, the compound, or a pharmaceutically acceptable salt thereof, is administered in a daily dose of about 30 mg/m² to about 80 mg/m² as measured by the amount of the free form of the compound. In some embodiments, the compound, or a pharmaceutically acceptable salt thereof, is administered in a daily dose of about 30 mg/m³ to about 60 mg/m² as measured by the amount of the free form of the compound. In some embodiments. the compound, or a pharmaceutically acceptable salt thereof, is administered in a daily dose of about 30 mg/m² to about 45 mg/m² as measured by the amount of the free form of the compound.

Provided herein is a method of treating cancer in a patient, comprising administering to said patient the compound, wherein the compound is administered in a daily dose of about 5 mg/m² to about 100 mg/m². In some embodiments, the compound is administered in a daily dose of about 10 mg/m² to about 100 mg/m². In some embodiments, the compound is administered in a daily dose of about 5 mg/m² to about 80 mg/m² In some embodiments, the compound is administered in a daily dose of about 10 mg/m² to about 80 mg/m² as measured by the amount of the free form of the compound. In some embodiments, the compound is administered in a daily dose of about 20 mg/m² to about 60 mg/m². In some embodiments, the compound is administered in a daily dose of about 20 mg/m² to about 100 mg/m². In some embodiments, the compound is administered in a daily dose of about 20 mg/m² to about 45 mg/m². In some embodiments, the compound is administered in a daily dose of about 20 mg/m² to about 30 mg/m². In some embodiments. the compound is administered in a daily dose of about 10 mg/m² to about 60 mg/m². In some embodiments, the compound is administered in a daily dose of about 10 mg/m² to about 45 mg/m². In some embodiments, the compound is administered in a daily dose of about 30 mg/m² to about 80 mg/m². In some embodiments, the compound is administered in a daily dose of about 30 mg/m² to about 60 mg/m². In some embodiments, the compound is administered in a daily dose of about 30 mg/m² to about 45 mg/m².

In some embodiments. the compound, or a pharmaceutically acceptable salt thereof, is administered in a daily dose of about 5 mg/m² as measured by the amount of the free form of the compound. In some embodiments, the compound, or a pharmaceutically acceptable salt thereof, is administered in a daily dose of about 10 mg/m² as measured by the amount of the free form of the compound. In some embodiments. the compound, or a pharmaceutically acceptable salt thereof, is administered in a daily dose of about 20 mg/m² as measured by the amount of the free form of the compound. In some embodiments, the compound, or a pharmaceutically acceptable salt thereof, is administered in a daily dose of about 30 mg/m² as measured by the amount of the free form of the compound. In some embodiments, the compound, or a pharmaceutically acceptable salt thereof, is administered in a daily dose of about 45 mg/m² as measured by the amount of the free form of the compound. In some embodiments, the compound, or a pharmaceutically acceptable salt thereof, is administered in a daily dose of about 60 mg/m² as measured by the amount of the free form of the compound.

In some embodiments, the compound, is administered in a daily dose of about 5 mg/m². In some embodiments, the compound is administered in a daily dose of about 10 mg/m². In some embodiments, the compound is administered in a daily dose of about 20 mg/m². In some embodiments, the compound is administered in a daily dose of about 30 mg/m². In some embodiments, the compound is administered in a daily dose of about 45 mg/m². In some embodiments, the compound is administered in a daily dose of about 60 mg/m².

In some embodiments, the compound, or a pharmaceutically acceptable salt thereof, is administered in a daily dose of about 10 mg/m² or less as measured by the amount of the free form of the compound. In some embodiments, the compound, or a pharmaceutically acceptable salt thereof, is administered in a daily dose of about 20 mg/m² or less as measured by the amount of the free form of the compound. In some embodiments, the compound, or a pharmaceutically acceptable salt thereof, is administered in a daily dose of about 30 mg/m² or less as measured by the amount of the free form of the compound. In some embodiments, the compound, or a pharmaceutically acceptable salt thereof, is administered in a daily dose of about 45 mg/m² or less as measured by the amount of the free form of the compound. In some embodiments. the compound, or a pharmaceutically acceptable salt thereof, is administered in a daily dose of about 60 mg/m² or less as measured by the amount of the free form of the compound.

In some embodiments, the compound is administered in a daily dose of about 5 mg/m² or less. In some embodiments. the compound is administered in a daily dose of about 10 mg/m² or less. In some embodiments, the compound is administered in a daily dose of about 20 mg/m² or less. In some embodiments, the compound is administered in a daily dose of about 30 mg/m² or less. In some embodiments, the compound is administered in a daily dose of about 45 mg/m² or less. In some embodiments, the compound is administered in a daily dose of about 60 mg/m² or less.

In some embodiments, the compound, or the pharmaceutically acceptable salt thereof, is administered as an intravenous infusion in a dosage of about 5 mg/m² to about 100 mg/m² as measured by the amount of the free form of the compound. In some embodiments, the compound, or a pharmaceutically acceptable salt thereof, is administered as an intravenous infusion in a dosage of about 10 mg/m² to about 100 mg/m² as measured by the amount of the free form of the compound. In some embodiments. the compound, or a pharmaceutically acceptable salt thereof, is administered as an intravenous infusion in a dosage of about 5 mg/m² to about 80 mg/m² as measured by the amount of the free form of the compound. In some embodiments, the compound, or a pharmaceutically acceptable salt thereof, is administered as an intravenous infusion in a dosage of about 10 mg/m² to about 80 mg/m² as measured by the amount of the free form of the compound. In some embodiments, the compound, or a pharmaceutically acceptable salt thereof, is administered as an intravenous infusion in a dosage of about 20 mg/m² to about 60 mg/m² as measured by the amount of the free form of the compound. In some embodiments, the compound, or a pharmaceutically acceptable salt thereof, is administered as an intravenous infusion in a dosage of about 20 mg/m² to about 100 mg/m² as measured by the amount of the free form of the compound. In some embodiments, the compound, or a pharmaceutically acceptable salt thereof, is administered as an intravenous infusion in a dosage of about 20 mg/m² to about 45 mg/m² as measured by the amount of the free form of the compound. In some embodiments, the compound, or a pharmaceutically acceptable salt thereof, is administered as an intravenous infusion in a dosage of about 20 mg/m² to about 30 mg/m² as measured by the amount of the free form of the compound. In some embodiments, the compound, or a pharmaceutically acceptable salt thereof, is administered as an intravenous infusion in a dosage of about 10 mg/m² to about 60 mg/m² as measured by the amount of the free form of the compound. In some embodiments. the compound, or a pharmaceutically acceptable salt thereof, is administered as an intravenous infusion in a dosage of about 10 mg/m² to about 45 mg/m² as measured by the amount of the free form of the compound. In some embodiments, the compound, or a pharmaceutically acceptable salt thereof, is administered as an intravenous infusion in a dosage of about 30 mg/m² to about 80 mg/m² as measured by the amount of the free form of the compound. In some embodiments, the compound, or a pharmaceutically acceptable salt thereof, is administered as an intravenous infusion in a dosage of about 30 mg/m² to about 60 mg/m² as measured by the amount of the free form of the compound In some embodiments, the compound, or a pharmaceutically acceptable salt thereof, is administered as an intravenous infusion in a dosage of about 30 mg/m² to about 45 mg/m² as measured by the amount of the free form of the compound.

Provided herein is a method of treating cancer in a patient, comprising administering to said patient the compound, wherein the compound is administered as an intravenous infusion in a dosage of about 5 mg/m² to about 100 mg/m². In some embodiments. the compound is administered as an intravenous infusion in a dosage of about 10 mg/m² to about 100 mg/m². In some embodiments, the compound is administered as an intravenous infusion in a dosage of about 5 mg/m² to about 80 mg/m². In some embodiments, the compound is administered as an intravenous infusion in a dosage of about 10 mg/m² to about 80 mg/m² as measured by the amount of the free form of the compound. In some embodiments, the compound is administered as an intravenous infusion in a dosage of about 20 mg/m² to about 60 mg/m². In some embodiments, the compound is administered as an intravenous infusion in a dosage of about 20 mg/m² to about 100 mg/m². In some embodiments, the compound is administered as an intravenous infusion in a dosage of about 20 mg/m² to about 45 mg/m². In some embodiments, the compound is administered as an intravenous infusion in a dosage of about 20 mg/m² to about 30 mg/m². In some embodiments. the compound is administered as an intravenous infusion in a dosage of about 10 mg/m² to about 60 mg/m². In some embodiments, the compound is administered as an intravenous infusion in a dosage of about 10 mg/m² to about 45 mg/m². In some embodiments, the compound is administered as an intravenous infusion in a dosage of about 30 mg/m² to about 80 mg/m². In some embodiments. the compound is administered as an intravenous infusion in a dosage of about 30 mg/m² to about 60 mg/m². In some embodiments, the compound is administered as an intravenous infusion in a dosage of about 30 mg/m² to about 45 mg/m².

In some embodiments, the compound, or a pharmaceutically acceptable salt thereof. is administered in a continuous dosing schedule. In some embodiments, the compound, or a pharmaceutically acceptable salt thereof, is administered once weekly.

In some embodiments. the compound, or a pharmaceutically acceptable salt thereof, is administered in an intermittent dosing schedule comprising one or more cycles, wherein each cycle comprises a first period of consecutive days wherein the compound, or the pharmaceutically acceptable salt thereof, is administered and a second period of consecutive days wherein the compound, or the pharmaceutically acceptable salt thereof, is not administered.

In some embodiments, the total length of each cycle is 7 days to 60 days. In some embodiments, the total length of each cycle is 14 days to 30 days. In some embodiments, the total length of each cycle is 21 days. In some embodiments, the total length of each cycle is 14 days. In some embodiments, the total length of each cycle is 28 days.

In some embodiments, the first period is 6 days and the second period is the remainder of the cycle. In some embodiments, the first period is 5 days and the second period is the remainder of the cycle. In some embodiments. the first period is 4 days and the second period is the remainder of the cycle. In some embodiments, the first period is 3 days and the second period is the remainder of the cycle. In some embodiments, the first period is 2 days and the second period is the remainder of the cycle. In some embodiments. the first period is 1 day and the second period is the remainder of the cycle.

In some embodiments, the compound, or a pharmaceutically acceptable salt thereof, is administered only on the first day of a cycle that is 21 days. In some embodiments. the compound, or a pharmaceutically acceptable salt thereof, is administered only on the first and the eighth day of a cycle that is 21 days. In some embodiments, the compound, or a pharmaceutically acceptable salt thereof, is administered only on the first, eighth, and fifteenth day of a cycle that is 21 days.

In some embodiments. the compound, or a pharmaceutically acceptable salt thereof, is administered only on the first day of a cycle that is 28 days. In some embodiments, the compound, or a pharmaceutically acceptable salt thereof, is administered only on the first and the eighth day of a cycle that is 28 days. In some embodiments, the compound, or a pharmaceutically acceptable salt thereof, is administered only on the first, eighth, and fifteenth day of a cycle that is 28 days. In some embodiments, the compound, or a pharmaceutically acceptable salt thereof, is administered only on the first, eighth, fifteenth, and twenty-second day of a cycle that is 28 days.

### Administration of Compound 1

The present application provides, inter alia, a method of treating cancer in a patient, comprising administering Compound 1, having the structure:

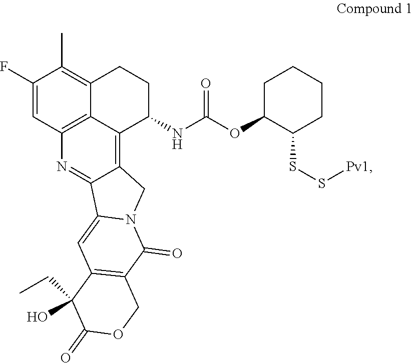

or a pharmaceutically acceptable salt thereof, wherein Pv1 is a peptide.

The present application further provides a method of administering Compound 1, or a pharmaceutically acceptable salt thereof, to a patient in need of treatment, comprising parenterally delivering to said patient Compound 1 having the structure:

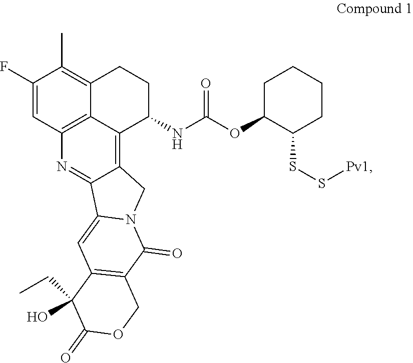

or a pharmaceutically acceptable salt thereof, wherein Pv1 is a peptide.

Compound 1 is described in US Patent Application Publication No. US 2021/009719, the entirety of which is incorporated herein by reference. Compound 1 is a peptide conjugate of exatecan, which is a topoisomerase I inhibitor. As a peptide conjugate of a topoisomerase I inhibitor, Compound 1 is useful in the treatment of various diseases such as cancer.

In some embodiments, Pv1 is a peptide comprising a 10-50 amino acid sequence, made up of naturally-occurring amino acid residues and optionally one or more non-naturally-occurring amino acids. In some embodiments, Pv1 is a peptide of 20 to 40, 20 to 30 amino acids, or 30 to 40 residues. In some embodiments, Pv1 is an environmentally sensitive peptide. As used herein, “environmentally sensitive peptides” are those that can insert across a cell membrane via a conformational change or a change in secondary structure in response to environmental (e.g., pH) changes. In some embodiments, the peptide is capable of selectively delivering the exatecan moiety across a cell membrane having an acidic or hypoxic mantle having a pH less than about 6.0.

In some embodiments, Pv1 is a peptide comprising the following sequence:

(SEQ ID NO: 1)
ADDQNPWRAYLDLLFPTDTLLLDLLWCG.

In some embodiments, Pv1 is a peptide comprising the following sequence:

(SEQ ID NO: 1)
ADDQNPWRAYLDLLFPTDTLLLDLLWCG,

wherein the sequence contains 1, 2, 3, 4, or 5 amino acid replacements, additions, or deletions.

In some embodiments, Pv1 is an environmentally sensitive peptide comprising the following sequence:

(SEQ ID NO: 1)
ADDQNPWRAYLDLLFPTDTLLLDLLWCG,

wherein the sequence contains 1, 2, 3, 4, or 5 amino acid replacements, additions, or deletions, so long as the peptide retains its environmental sensitivity.

In some embodiments, Pv1 is a peptide consisting essentially of the following sequence:

(SEQ ID NO: 1)
ADDQNPWRAYLDLLFPTDTLLLDLLWCG.

In some embodiments, Pv1 is a peptide consisting of the following sequence:

(SEQ ID NO: 1)
ADDQNPWRAYLDLLFPTDTLLLDLLWCG.

Provided herein is a method of treating cancer in a patient, comprising administering to said patient Compound 1, or a pharmaceutically acceptable salt thereof, wherein Compound 1, or the pharmaceutically acceptable salt thereof, is administered in a daily dose of about 0.1 mg/kg to about 1.5 mg/kg as measured by the amount of the free form of Compound 1. In some embodiments. Compound 1, or a pharmaceutically acceptable salt thereof, is administered in a daily dose of about 0.25 mg/kg to about 1.5 mg/kg as measured by the amount of the free form of Compound 1. In some embodiments, Compound 1, or a pharmaceutically acceptable salt thereof, is administered in a daily dose of about 0.1 mg/kg to 1.25 mg/kg as measured by the amount of the free form of Compound 1. In some embodiments, Compound 1, or a pharmaceutically acceptable salt thereof, is administered in a daily dose of about 0.25 mg/kg to about 1.25 mg/kg as measured by the amount of the free form of Compound 1. In some embodiments, Compound 1, or a pharmaceutically acceptable salt thereof, is administered in a daily dose of about 0.25 mg/kg to about 0.75 mg/kg as measured by the amount of the free form of Compound 1. In some embodiments, Compound 1, or a pharmaceutically acceptable salt thereof, is administered in a daily dose of about 0.25 mg/kg to about 0.50 mg/kg as measured by the amount of the free form of Compound 1. In some embodiments, Compound 1, or a pharmaceutically acceptable salt thereof, is administered in a daily dose of about 0.5 mg/kg to about 0.75 mg/kg as measured by the amount of the free form of Compound 1. In some embodiments. Compound 1, or a pharmaceutically acceptable salt thereof, is administered in a daily dose of about 0.5 mg/kg to about 1.25 mg/kg as measured by the amount of the free form of Compound 1.

Provided herein is a method of treating cancer in a patient, comprising administering to said patient Compound 1, wherein Compound 1 is administered in a daily dose of about 0.1 mg/kg to about 1.5 mg/kg. In some embodiments, Compound 1 is administered in a daily dose of about 0.25 mg/kg to about 1.5 mg/kg. In some embodiments, Compound 1 is administered in a daily dose of about 0.1 mg/kg to 1.25 mg/kg. In some embodiments. Compound 1 is administered in a daily dose of about 0.25 mg/kg to about 1.25 mg/kg. In some embodiments, Compound 1 is administered in a daily dose of about 0.25 mg/kg to about 0.75 mg/kg. In some embodiments, Compound 1 is administered in a daily dose of about 0.25 mg/kg to about 0.50 mg/kg. In some embodiments. Compound 1 is administered in a daily dose of about 0.5 mg/kg to about 0.75 mg/kg. In some embodiments. Compound 1 is administered in a daily dose of about 0.5 mg/kg to about 1.25 mg/kg.

In some embodiments. Compound 1, or a pharmaceutically acceptable salt thereof, is administered in a daily dose of about 0.25 mg/kg as measured by the amount of the free form of Compound 1. In some embodiments, Compound 1, or a pharmaceutically acceptable salt thereof, is administered in a daily dose of about 0.5 mg/kg as measured by the amount of the free form of Compound 1. In some embodiments. Compound 1, or a pharmaceutically acceptable salt thereof, is administered in a daily dose of about 0.75 mg/kg as measured by the amount of the free form of Compound 1. In some embodiments, Compound 1, or a pharmaceutically acceptable salt thereof, is administered in a daily dose of about 1.0 mg/kg as measured by the amount of the free form of Compound 1. In some embodiments, Compound 1, or a pharmaceutically acceptable salt thereof, is administered in a daily dose of about 1.25 mg/kg as measured by the amount of the free form of Compound 1.

In some embodiments, Compound 1, is administered in a daily dose of about 0.25 mg/kg. In some embodiments, Compound 1 is administered in a daily dose of about 0.5 mg/kg. In some embodiments, Compound 1 is administered in a daily dose of about 0.75 mg/kg. In some embodiments, Compound 1 is administered in a daily dose of about 1.0 mg/kg. In some embodiments. Compound 1 is administered in a daily dose of about 1.25 mg/kg.

In some embodiments, Compound 1, or a pharmaceutically acceptable salt thereof, is administered in a daily dose of about 1.5 mg/kg or less as measured by the amount of the free form of Compound 1. In some embodiments, Compound 1, or a pharmaceutically acceptable salt thereof, is administered in a daily dose of about 1.25 mg/kg or less as measured by the amount of the free form of Compound 1. In some embodiments, Compound 1, or a pharmaceutically acceptable salt thereof, is administered in a daily dose of about 1.0 mg/kg or less as measured by the amount of the free form of Compound 1. In some embodiments, Compound 1, or a pharmaceutically acceptable salt thereof, is administered in a daily dose of about 0.75 mg/kg or less as measured by the amount of the free form of Compound 1. In some embodiments, Compound 1, or a pharmaceutically acceptable salt thereof, is administered in a daily dose of about 0.5 mg/kg or less as measured by the amount of the free form of Compound 1. In some embodiments, Compound 1, or a pharmaceutically acceptable salt thereof, is administered in a daily dose of about 0.25 mg/kg or less as measured by the amount of the free form of Compound 1.

In some embodiments, Compound 1 is administered in a daily dose of about 1.5 mg/kg or less. In some embodiments, Compound 1 is administered in a daily dose of about 1.25 mg/kg or less. In some embodiments. Compound 1 is administered in a daily dose of about 1.0 mg/kg or less. In some embodiments, Compound 1 is administered in a daily dose of about 0.75 mg/kg or less. In some embodiments, Compound 1 is administered in a daily dose of about 0.5 mg/kg or less. In some embodiments, Compound 1 is administered in a daily dose of about 0.25 mg/kg or less.

In some embodiments, Compound 1, or a pharmaceutically acceptable salt thereof, is administered intravenously. In some embodiments, Compound 1, or a pharmaceutically acceptable salt thereof, is administered as an intravenous infusion. In some embodiments, Compound 1, or a pharmaceutically acceptable salt thereof, is administered as an intravenous infusion in a dosage of about 0.1 mg/kg to about 1.5 mg/kg as measured by the amount of the free form of Compound 1. In some embodiments, Compound 1, or a pharmaceutically acceptable salt thereof, is administered as an intravenous infusion in a dosage of about 0.25 mg/kg to about 1.5 mg/kg as measured by the amount of the free form of Compound 1. In some embodiments, Compound 1, or a pharmaceutically acceptable salt thereof, is administered as an intravenous infusion in a dosage of about 0.1 mg/kg to about 1.25 mg/kg as measured by the amount of the free form of Compound 1. In some embodiments, Compound 1, or a pharmaceutically acceptable salt thereof, is administered as an intravenous infusion in a dosage of about 0.25 mg/kg to about 1.25 mg/kg as measured by the amount of the free form of Compound 1. In some embodiments. Compound 1, or a pharmaceutically acceptable salt thereof, is administered as an intravenous infusion in a dosage of about 0.25 mg/kg to about 0.75 mg/kg as measured by the amount of the free form of Compound 1. In some embodiments, Compound 1, or a pharmaceutically acceptable salt thereof, is administered as an intravenous infusion in a dosage of about 0.25 mg/kg to about 0.5 mg/kg as measured by the amount of the free form of Compound 1. In some embodiments,

Compound 1, or a pharmaceutically acceptable salt thereof, is administered as an intravenous infusion in a dosage of about 0.5 mg/kg to about 0.75 mg/kg as measured by the amount of the free form of Compound 1. In some embodiments. Compound 1, or a pharmaceutically acceptable salt thereof, is administered as an intravenous infusion in a dosage of about 0.5 mg/kg to about 1.25 mg/kg as measured by the amount of the free form of Compound 1. In some embodiments. Compound 1, or a pharmaceutically acceptable salt thereof, is administered as an intravenous infusion in a dosage of about 0.5 mg/kg to about 1.5 mg/kg as measured by the amount of the free form of Compound 1. In some embodiments. Compound 1, or a pharmaceutically acceptable salt thereof, is administered as an intravenous infusion in a dosage of about 0.25 mg/kg as measured by the amount of the free form of Compound 1. In some embodiments, Compound 1, or a pharmaceutically acceptable salt thereof, is administered as an intravenous infusion in a dosage of about 0.5 mg/kg as measured by the amount of the free form of Compound 1. In some embodiments. Compound 1, or a pharmaceutically acceptable salt thereof, is administered as an intravenous infusion in a dosage of about 0.75 mg/kg as measured by the amount of the free form of Compound 1. In some embodiments, Compound 1, or a pharmaceutically acceptable salt thereof, is administered as an intravenous infusion in a dosage of about 1.0 mg/kg as measured by the amount of the free form of Compound 1. In some embodiments, Compound 1, or a pharmaceutically acceptable salt thereof, is administered as an intravenous infusion in a dosage of about 1.25 mg/kg as measured by the amount of the free form of Compound 1.

In some embodiments, Compound 1 is administered intravenously. In some embodiments, Compound 1 is administered as an intravenous infusion. In some embodiments, Compound 1 is administered as an intravenous infusion in a dosage of about 0.1 mg/kg to about 1.5 mg/kg. In some embodiments, Compound 1 is administered as an intravenous infusion in a dosage of about 0.25 mg/kg to about 1.5 mg/kg. In some embodiments. Compound 1 is administered as an intravenous infusion in a dosage of about 0.1 mg/kg to about 1.25 mg/kg. In some embodiments, Compound 1 is administered as an intravenous infusion in a dosage of about 0.25 mg/kg to about 1.25 mg/kg. In some embodiments, Compound 1 is administered as an intravenous infusion in a dosage of about 0.25 mg/kg to about 0.75 mg/kg. In some embodiments, Compound 1 is administered as an intravenous infusion in a dosage of about 0.25 mg/kg to about 0.5 mg/kg. In some embodiments. Compound 1 is administered as an intravenous infusion in a dosage of about 0.5 mg/kg to about 0.75 mg/kg. In some embodiments, Compound 1 is administered as an intravenous infusion in a dosage of about 0.5 mg/kg to about 1.25 mg/kg. In some embodiments. Compound 1 is administered as an intravenous infusion in a dosage of about 0.5 mg/kg to about 1.5 mg/kg. In some embodiments, Compound 1 is administered as an intravenous infusion in a dosage of about 0 25 mg/kg. In some embodiments, Compound 1 is administered as an intravenous infusion in a dosage of about 0.5 mg/kg. In some embodiments, Compound 1 is administered as an intravenous infusion in a dosage of about 0.75 mg/kg. In some embodiments. Compound 1 is administered as an intravenous infusion in a dosage of about 1.0 mg/kg. In some embodiments. Compound 1 is administered as an intravenous infusion in a dosage of about 1.25 mg/kg.

Provided herein is a method of treating cancer in a patient, comprising administering to said patient Compound 1, or a pharmaceutically acceptable salt thereof, wherein Compound 1, or the pharmaceutically acceptable salt thereof, is administered in a daily dose of about 5 mg/m² to about 100 mg/m² as measured by the amount of the free form of Compound 1. In some embodiments. Compound 1, or a pharmaceutically acceptable salt thereof, is administered in a daily dose of about 10 mg/m² to about 100 mg/m² as measured by the amount of the free form of Compound 1. In some embodiments, Compound 1, or a pharmaceutically acceptable salt thereof, is administered in a daily dose of about 5 mg/m² to about 80 mg/m² as measured by the amount of the free form of Compound 1. In some embodiments, Compound 1, or a pharmaceutically acceptable salt thereof, is administered in a daily dose of about 10 mg/m² to about 80 mg/m² as measured by the amount of the free form of Compound 1. In some embodiments, Compound 1, or a pharmaceutically acceptable salt thereof, is administered in a daily dose of about 20 mg/m² to about 60 mg/m² as measured by the amount of the free form of Compound 1. In some embodiments, Compound 1, or a pharmaceutically acceptable salt thereof, is administered in a daily dose of about 20 mg/m² to about 100 mg/m² as measured by the amount of the free form of Compound 1. In some embodiments, Compound 1, or a pharmaceutically acceptable salt thereof, is administered in a daily dose of about 20 mg/m² to about 45 mg/m² as measured by the amount of the free form of Compound 1. In some embodiments. Compound 1, or a pharmaceutically acceptable salt thereof, is administered in a daily dose of about 20 mg/m² to about 30 mg/m² as measured by the amount of the free form of Compound 1. In some embodiments. Compound 1, or a pharmaceutically acceptable salt thereof, is administered in a daily dose of about 10 mg/m² to about 60 mg/m² as measured by the amount of the free form of Compound 1. In some embodiments. Compound 1, or a pharmaceutically acceptable salt thereof, is administered in a daily dose of about 10 mg/m² to about 45 mg/m² as measured by the amount of the free form of Compound 1. In some embodiments, Compound 1, or a pharmaceutically acceptable salt thereof, is administered in a daily dose of about 30 mg/m² to about 80 mg/m² as measured by the amount of the free form of Compound 1. In some embodiments, Compound 1, or a pharmaceutically acceptable salt thereof, is administered in a daily dose of about 30 mg/m² to about 60 mg/m² as measured by the amount of the free form of Compound 1. In some embodiments. Compound 1, or a pharmaceutically acceptable salt thereof, is administered in a daily dose of about 30 mg/m² to about 45 mg/m² as measured by the amount of the free form of Compound 1.

Provided herein is a method of treating cancer in a patient, comprising administering to said patient Compound 1, wherein Compound 1 is administered in a daily dose of about 5 mg/m² to about 100 mg/m². In some embodiments, Compound 1 is administered in a daily dose of about 10 mg/m² to about 100 mg/m². In some embodiments, Compound 1 is administered in a daily dose of about 5 mg/m² to about 80 mg/m². In some embodiments, Compound 1 is administered in a daily dose of about 10 mg/m² to about 80 mg/m² as measured by the amount of the free form of Compound 1. In some embodiments, Compound 1 is administered in a daily dose of about 20 mg/m² to about 60 mg/m². In some embodiments. Compound 1 is administered in a daily dose of about 20 mg/m² to about 100 mg/m². In some embodiments, Compound 1 is administered in a daily dose of about 20 mg/m² to about 45 mg/m². In some embodiments, Compound 1 is administered in a daily dose of about 20 mg/m² to about 30 mg/m². In some embodiments, Compound 1 is administered in a daily dose of about 10 mg/m² to about 60 mg/m². In some embodiments. Compound 1 is administered in a daily dose of about 10 mg/m² to about 45 mg/m². In some embodiments, Compound 1 is administered in a daily dose of about 30 mg/m² to about 80 mg/m². In some embodiments. Compound 1 is administered in a daily dose of about 30 mg/m² to about 60 mg/m². In some embodiments, Compound 1 is administered in a daily dose of about 30 mg/m² to about 45 mg/m².

In some embodiments, Compound 1, or a pharmaceutically acceptable salt thereof, is administered in a daily dose of about 5 mg/m² as measured by the amount of the free form of Compound 1. In some embodiments, Compound 1, or a pharmaceutically acceptable salt thereof, is administered in a daily dose of about 10 mg/m² as measured by the amount of the free form of Compound 1. In some embodiments, Compound 1, or a pharmaceutically acceptable salt thereof, is administered in a daily dose of about 20 mg/m² as measured by the amount of the free form of Compound 1. In some embodiments, Compound 1, or a pharmaceutically acceptable salt thereof, is administered in a daily dose of about 30 mg/m² as measured by the amount of the free form of Compound 1. In some embodiments, Compound 1, or a pharmaceutically acceptable salt thereof, is administered in a daily dose of about 45 mg/m² as measured by the amount of the free form of Compound 1. In some embodiments, Compound 1, or a pharmaceutically acceptable salt thereof, is administered in a daily dose of about 60 mg/m² as measured by the amount of the free form of Compound 1.

In some embodiments, Compound 1 is administered in a daily dose of about 5 mg/m². In some embodiments, Compound 1 is administered in a daily dose of about 10 mg/m². In some embodiments, Compound 1 is administered in a daily dose of about 20 mg/m². In some embodiments, Compound 1 is administered in a daily dose of about 30 mg/m². In some embodiments, Compound 1 is administered in a daily dose of about 45 mg/m². In some embodiments, Compound 1 is administered in a daily dose of about 60 mg/m².

In some embodiments, Compound 1, or a pharmaceutically acceptable salt thereof, is administered in a daily dose of about 10 mg/m² or less as measured by the amount of the free form of Compound 1. In some embodiments. Compound 1, or a pharmaceutically acceptable salt thereof, is administered in a daily dose of about 20 mg/m² or less as measured by the amount of the free form of Compound 1. In some embodiments, Compound 1, or a pharmaceutically acceptable salt thereof, is administered in a daily dose of about 30 mg/m² or less as measured by the amount of the free form of Compound 1. In some embodiments, Compound 1, or a pharmaceutically acceptable salt thereof, is administered in a daily dose of about 45 mg/m² or less as measured by the amount of the free form of Compound 1. In some embodiments. Compound 1, or a pharmaceutically acceptable salt thereof, is administered in a daily dose of about 60 mg/m² or less as measured by the amount of the free form of Compound 1.

In some embodiments, Compound 1, is administered in a daily dose of about 5 mg/m² or less. In some embodiments. Compound 1 is administered in a daily dose of about 10 mg/m² or less. In some embodiments, Compound 1 is administered in a daily dose of about 20 mg/m² or less. In some embodiments, Compound 1 is administered in a daily dose of about 30 mg/m² or less. In some embodiments, Compound 1 is administered in a daily dose of about 45 mg/m² or less. In some embodiments, Compound 1 is administered in a daily dose of about 60 mg/m² or less.

In some embodiments. Compound 1, or a pharmaceutically acceptable salt thereof, is administered intravenously. In some embodiments, Compound 1, or a pharmaceutically acceptable salt thereof, is administered as an intravenous infusion. In some embodiments, Compound 1, or the pharmaceutically acceptable salt thereof, is administered as an intravenous infusion in a dosage of about 5 mg/m² to about 100 mg/m² as measured by the amount of the free form of Compound 1. In some embodiments, Compound 1, or a pharmaceutically acceptable salt thereof, is administered as an intravenous infusion in a dosage of about 10 mg/m² to about 100 mg/m² as measured by the amount of the free form of Compound 1. In some embodiments, Compound 1, or a pharmaceutically acceptable salt thereof, is administered as an intravenous infusion in a dosage of about 5 mg/m² to about 80 mg/m² as measured by the amount of the free form of Compound 1. In some embodiments, Compound 1, or a pharmaceutically acceptable salt thereof, is administered as an intravenous infusion in a dosage of about 10 mg/m² to about 80 mg/m² as measured by the amount of the free form of Compound 1. In some embodiments, Compound 1, or a pharmaceutically acceptable salt thereof, is administered as an intravenous infusion in a dosage of about 20 mg/m² to about 60 mg/m² as measured by the amount of the free form of Compound 1. In some embodiments, Compound 1, or a pharmaceutically acceptable salt thereof, is administered as an intravenous infusion in a dosage of about 20 mg/m² to about 100 mg/m² as measured by the amount of the free form of Compound 1. In some embodiments. Compound 1, or a pharmaceutically acceptable salt thereof, is administered as an intravenous infusion in a dosage of about 20 mg/m² to about 45 mg/m² as measured by the amount of the free form of Compound 1. In some embodiments, Compound 1, or a pharmaceutically acceptable salt thereof, is administered as an intravenous infusion in a dosage of about 20 mg/m² to about 30 mg/m² as measured by the amount of the free form of Compound 1. In some embodiments. Compound 1, or a pharmaceutically acceptable salt thereof, is administered as an intravenous infusion in a dosage of about 10 mg/m² to about 60 mg/m² as measured by the amount of the free form of Compound 1. In some embodiments. Compound 1, or a pharmaceutically acceptable salt thereof, is administered as an intravenous infusion in a dosage of about 10 mg/m² to about 45 mg/m² as measured by the amount of the free form of Compound 1. In some embodiments, Compound 1, or a pharmaceutically acceptable salt thereof, is administered as an intravenous infusion in a dosage of about 30 mg/m² to about 80 mg/m² as measured by the amount of the free form of Compound 1. In some embodiments. Compound 1, or a pharmaceutically acceptable salt thereof, is administered as an intravenous infusion in a dosage of about 30 mg/m² to about 60 mg/m² as measured by the amount of the free form of Compound 1. In some embodiments, Compound 1, or a pharmaceutically acceptable salt thereof, is administered as an intravenous infusion in a dosage of about 30 mg/m² to about 45 mg/m² as measured by the amount of the free form of Compound 1.

Provided herein is a method of treating cancer in a patient, comprising administering to said patient Compound 1, wherein Compound 1 is administered as an intravenous infusion in a dosage of about 5 mg/m² to about 100 mg/m². In some embodiments, Compound 1 is administered as an intravenous infusion in a dosage of about 10 mg/m² to about 100 mg/m². In some embodiments, Compound 1 is administered as an intravenous infusion in a dosage of about 5 mg/m² to about 80 mg/m². In some embodiments, Compound 1 is administered as an intravenous infusion in a dosage of about 10 mg/m² to about 80 mg/m² as measured by the amount of the free form of Compound 1. In some embodiments. Compound 1 is administered as an intravenous infusion in a dosage of about 20 mg/m² to about 60 mg/m². In some embodiments, Compound 1 is administered as an intravenous infusion in a dosage of about 20 mg/m² to about 100 mg/m². In some embodiments, Compound 1 is administered as an intravenous infusion in a dosage of about 20 mg/m² to about 45 mg/m². In some embodiments, Compound 1 is administered as an intravenous infusion in a dosage of about 20 mg/m² to about 30 mg/m². In some embodiments, Compound 1 is administered as an intravenous infusion in a dosage of about 10 mg/m² to about 60 mg/m². In some embodiments, Compound 1 is administered as an intravenous infusion in a dosage of about 10 mg/m² to about 45 mg/m². In some embodiments, Compound 1 is administered as an intravenous infusion in a dosage of about 30 mg/m² to about 80 mg/m². In some embodiments. Compound 1 is administered as an intravenous infusion in a dosage of about 30 mg/m² to about 60 mg/m². In some embodiments, Compound 1 is administered as an intravenous infusion in a dosage of about 30 mg/m² to about 45 mg/m².

In some embodiments, Compound 1, or a pharmaceutically acceptable salt thereof, is administered in a continuous dosing schedule. In some embodiments. Compound 1, or a pharmaceutically acceptable salt thereof, is administered once weekly.

In some embodiments, Compound 1, or a pharmaceutically acceptable salt thereof, is administered in an intermittent dosing schedule comprising one or more cycles, wherein each cycle comprises a first period of consecutive days wherein Compound 1, or the pharmaceutically acceptable salt thereof, is administered and a second period of consecutive days wherein Compound 1, or the pharmaceutically acceptable salt thereof, is not administered.

In some embodiments, the total length of each cycle is 7 days to 60 days. In some embodiments, the total length of each cycle is 14 days to 30 days. In some embodiments, the total length of each cycle is 21 days. In some embodiments, the total length of each cycle is 14 days. In some embodiments, the total length of each cycle is 28 days.

In some embodiments. the first period is 6 days and the second period is the remainder of the cycle. In some embodiments, the first period is 5 days and the second period is the remainder of the cycle. In some embodiments, the first period is 4 days and the second period is the remainder of the cycle. In some embodiments, the first period is 3 days and the second period is the remainder of the cycle. In some embodiments, the first period is 2 days and the second period is the remainder of the cycle. In some embodiments, the first period is 1 day and the second period is the remainder of the cycle.

In some embodiments. Compound 1, or a pharmaceutically acceptable salt thereof, is administered only on the first day of a cycle that is 21 days. In some embodiments. Compound 1, or a pharmaceutically acceptable salt thereof, is administered only on the first and the eighth day of a cycle that is 21 days. In some embodiments, Compound 1, or a pharmaceutically acceptable salt thereof, is administered only on the first, eighth, and fifteenth day of a cycle that is 21 days.

In some embodiments. Compound 1, or a pharmaceutically acceptable salt thereof, is administered only on the first day of a cycle that is 28 days. In some embodiments. Compound 1, or a pharmaceutically acceptable salt thereof, is administered only on the first and the eighth day of a cycle that is 28 days. In some embodiments, Compound 1, or a pharmaceutically acceptable salt thereof, is administered only on the first, eighth, and fifteenth day of a cycle that is 28 days. In some embodiments, Compound 1, or a pharmaceutically acceptable salt thereof, is administered only on the first, eighth, fifteenth, and twenty-second day of a cycle that is 28 days.

In some embodiments, the recited dosage is expressed in terms of a patient's body weight and is provided in units of mg/kg (e.g., amount of the compound to be administered divided by the patient's body weight). In some embodiments, the recited dosage is expressed in terms of a patient's body surface area and is provided in units of mg/m² (e.g., amount of the compound to be administered divided by the patient's body surface area squared). A patient's body surface area can be calculated using methods and formulae known to those of ordinary skilly in the art. For example. a patient's body surface arca can be calculated using the 1987 formula published by Mosteller (Mosteller RD. Simplified calculation of body-surface area. N Engl J Med. 1987; 317(17): 1098).

Example cancers that are treatable using the disclosed methods are ovarian cancer. small cell lung cancer (SCLC), non-small cell lung cancer (NSCLC), breast cancer, gastric cancer, esophageal cancer, colorectal cancer, pancreatic cancer, urothelial cancer, and sarcoma. Further examples of cancers that are treatable using the disclosed methods are appendiceal cancer and osteosarcoma.

Further examples of cancers that are treatable using the methods of the present disclosure include, but are not limited to, colorectal cancer, gastric cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina. carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma. pituitary adenoma, Kaposi's sarcoma, epidermoid cancer, squamous cell cancer, T-cell lymphoma, environmentally induced cancers including those induced by asbestos, and combinations of said cancers.

In some embodiments, cancers treatable with methods of the present disclosure include bladder cancer, bone cancer, glioma, breast cancer (e.g., triple-negative breast cancer), cervical cancer, colon cancer, colorectal cancer, endometrial cancer, epithelial cancer, esophageal cancer, Ewing's sarcoma, pancreatic cancer, gallbladder cancer, gastric cancer, gastrointestinal tumors, head and neck cancer (upper acrodigestive cancer), intestinal cancers, Kaposi's sarcoma, kidney cancer, laryngeal cancer, liver cancer (e.g., hepatocellular carcinoma), lung cancer (e.g., non-small cell lung cancer, adenocarcinoma), melanoma, prostate cancer, rectal cancer, renal clear cell carcinoma, skin cancer, stomach cancer, testicular cancer, thyroid cancer, and uterine cancer.

In some embodiments, cancers treatable with methods of the present disclosure include melanoma (e.g., metastatic malignant melanoma), renal cancer (e.g. clear cell carcinoma), prostate cancer (e.g. hormone refractory prostate adenocarcinoma), breast cancer, triple-negative breast cancer, colon cancer and lung cancer (e.g. non-small cell lung cancer and small cell lung cancer). Additionally, the disclosure includes refractory or recurrent malignancies whose growth may be inhibited using Compound 1 or another compound of the disclosure.

In some embodiments, cancers that are treatable using the methods of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer. cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL. mantle cell lymphoma. Non-Hodgkin lymphoma (including relapsed or refractory NHL and recurrent follicular), Hodgkin lymphoma or multiple myeloma) and combinations of said cancers.

The compounds of the disclosure can exhibit certain therapeutic advantages over a topoisomerase I inhibitor itself. For example, administration of a compound of the disclosure can show reduced toxicity (e.g., bone marrow or gastric toxicity) as compared with administration of the corresponding topoisomerase I inhibitor (e.g., exatecan). In some embodiments. the bone marrow toxicity is measured by total bone marrow count from samples of the subject (e.g., total bone marrow count in femurs of a mouse). In some embodiments, bone marrow toxicity is measured by PARylation in bone marrow tissue. In some embodiments. bone marrow toxicity is measured according to total nucleated bone marrow cells. In some embodiments, gastric toxicity is assessed using photographs of the stomachs of the subject (e.g., a mouse) taken both in situ and ex vivo.

Compound 1 can exhibit certain therapeutic advantages over a topoisomerase I inhibitor itself. For example, administration of Compound 1 can show reduced toxicity (e.g., bone marrow or gastric toxicity) as compared with administration of the corresponding topoisomerase I inhibitor (e.g., exatecan). In some embodiments, the bone marrow toxicity is measured by total bone marrow count from samples of the subject (e.g., total bone marrow count in femurs of a mouse). In some embodiments, bone marrow toxicity is measured by PARylation in bone marrow tissue. In some embodiments, bone marrow toxicity is measured according to total nucleated bone marrow cells. In some embodiments, gastric toxicity is assessed using photographs of the stomachs of the subject (e.g., a mouse) taken both in situ and ex vivo.

In some embodiments. the cancer is refractory. In some embodiments. the patient has failed at least one previous treatment for cancer. The previous treatment for cancer can include, for example, a chemotherapeutic agent, a targeted cancer therapy, an immunotherapy or radiation therapy. The chemotherapeutic agent of a previous treatment can include any exemplary chemotherapeutic agent listed herein.

In some embodiments, a compound of Formula (I), or a pharmaceutically acceptable salt thereof, is administered in combination with an additional therapy. The additional therapy can include, for example, a chemotherapeutic agent, a targeted cancer therapy, an immunotherapy or radiation therapy.

In some embodiments, Compound 1, or a pharmaceutically acceptable salt thereof, is administered in combination with an additional therapy. The additional therapy can include, for example, a chemotherapeutic agent, a targeted cancer therapy, an immunotherapy or radiation therapy.

Exemplary chemotherapeutic agents include, for example, alkylating agents (including, without limitation, nitrogen mustards, ethylenimine derivatives, alkyl sulfonates, nitrosourcas and triazenes) such as uracil mustard, chlormethine, cyclophosphamide (CytoxanTM), ifosfamide, melphalan, chlorambucil, pipobroman, triethylene-melamine, tricthylenethio phosphoramine, busulfan, carmustine, lomustine, streptozocin, dacarbazine. and temozolomide.

Further chemotherapeutic agents include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC: or temozolomide.

Further chemotherapeutic agents include: immunotherapy drugs, including cytokines such as interferon alpha, interleukin 2, and tumor necrosis factor (TNF).

Further chemotherapeutic agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Further chemotherapeutic agents include, for example, certain natural products and their derivatives (for example, vinca alkaloids, antitumor antibiotics, enzymes, lymphokines and epipodophyllotoxins) such as vinblastine, vincristine, vindesine, bleomycin. dactino-mycin, daunorubicin, doxorubicin, epirubicin, idarubicin, ara-C, paclitaxel (TAXOLTM), mithramycin, deoxycoformycin, mitomycin-C, L-asparaginase, interferons (especially IFN-a), etoposide, and teniposide.

Further chemotherapeutic agents include, for example, navelbene. CPT-11, anastrazole, letrazole, capecitabine, reloxafine, cyclophosphamide, ifosamide, and droloxafine.

Further chemotherapeutic agents include, for example, epidophyllotoxin; an antineoplastic enzyme: a topoisomerase inhibitor: procarbazine; mitoxantrone: platinum coordination complexes such as cis-platin and carboplatin: biological response modifiers: growth inhibitors; antihormonal therapeutic agents; leucovorin: tegafur; and haematopoietic growth factors.

Further chemotherapeutic agents include antibody therapeutics such as trastuzumab (Herceptin), antibodies to costimulatory molecules such as CTLA-4, 4-1BB and PD-1, or antibodies to cytokines (IL-10, TGF-α, etc.).

Further chemotherapeutic agents include those that block immune cell migration such as antagonists to chemokine receptors, including CCR2 and CCR4.

Further chemotherapeutic agents include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles (Abraxane®).

The term “treating” as used herein includes the administration of a compound or composition which reduces the frequency of, delays the onset of, or reduces the progression of symptoms of a disease involving acidic or hypoxic diseased tissue, such as cancer, stroke, myocardial infarction, or long-term neurodegenerative disease, in a subject relative to a subject not receiving the compound or composition. This can include reversing, reducing, or arresting the symptoms, clinical signs, or underlying pathology of a condition in a manner to improve or stabilize a subject's condition (e.g., regression of tumor growth, for cancer or decreasing or ameliorating myocardial ischemia reperfusion injury in myocardial infarction, stroke, or the like cardiovascular disease). The terms “inhibiting” or “reducing” are used for cancer in reference to methods to inhibit or to reduce tumor growth (e.g., decrease the size of a tumor) in a population as compared to an untreated control population.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines: alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two: generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences. 171^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002).

All publications (including patents) mentioned herein are incorporated herein by reference for the purpose of describing and disclosing, for example, the constructs and methodologies that are described in the publications, which might be used in connection with the disclosure herein described. The publications discussed throughout the text are provided solely for their disclosure prior to the filing date of the present application.

Disclosed herein are several types of ranges. When a range of any type is disclosed or claimed, the intent is to disclose or claim individually each possible number that such a range could reasonably encompass, including end points of the range as well as any sub-ranges and combinations of sub-ranges encompassed therein. When a range of therapeutically effective amounts of an active ingredient is disclosed or claimed, for instance, the intent is to disclose or claim individually every possible number that such a range could encompass, consistent with the disclosure herein. For example, by a disclosure that the therapeutically effective amount of a compound can be in a range from about 1 mg/kg to about 50 mg/kg (of body weight of the subject).

### EXAMPLES

### Example 1

### Phase 1/2 Study of Compound 1

This is a first-in-human, Phase 1/2 open-label, multicenter, dose-escalation, safety, pharmacokinetics (PK), and biomarker study of Compound 1 in subjects with advanced or metastatic refractory solid tumors.

Phase I is the dose-escalation portion of the study in which the safety and tolerability of two dosing schedules of Compound 1 will be evaluated.

### Primary Objectives:

To determine the safety and tolerability, maximum tolerated doses (MTDs) and dose limiting toxicities (DLTs) of Compound 1 in subjects with advanced or metastatic refractory solid tumors when administered on the following schedules:
   Daily×5 every 3 weeks (Part A)
   Daily×3 every 3 weeks (Part B)
To establish the Recommended Phase 2 Dose (RP2D) of Compound 1 for each schedule

### Secondary Objectives:

To determine the pharmacokinetics (PK) of Compound 1
To evaluate the preliminary anti-tumor activity of Compound 1 based on Response Evaluation Criteria in Solid Tumors (RECIST) 1.1 as follows:
   Objective response rate (ORR)
   Duration of response (DoR)
   Progression-free survival (PFS)

### Exploratory Objectives

To evaluate the development and impact of anti-drug antibodies (ADAs)
To evaluate the PK of unconjugated exatecan
To evaluate potential biomarkers of Compound 1 (e.g., intra-tumoral concentrations of exatecan)

### Part A

Phase 1 Part A will open for accrual before Part B. Subjects in Part A are treated with Compound 1 daily. 5 times per week every 3 weeks (Schedule A. i.e., 5 days on, 16 days off. Cycle length is 3 weeks). A 3+3 design is utilized. However, in order to minimize the number of subjects treated at potentially sub-therapeutic doses, the initial cohorts in Phase 1 will enroll a single subject. Single-subject cohorts will be enrolled until a subject has a Grade 2 Adverse Event (AE) considered possibly related to Compound 1 during the dose-limiting toxicity (DLT) period (i.e., the subject's initial 3-weck cycle), at which time 2 additional subjects will be enrolled in that cohort, and a 3+3 design will subsequently be utilized. Dose escalations of up to 100% of the prior dose are permitted until the occurrence of a Grade 2 AE considered possibly related to Compound 1 during the DLT period.

Subsequent dose escalations may be no more than 50% of the prior dose.

Under the 3+3 design, cohorts of three subjects will enroll at a dose level. If none of the three subjects experience a DLT, the dose will be escalated to the next highest dose level. If one of the three initial subjects in a cohort experiences a DLT, up to three additional subjects will be enrolled and treated at the same dose. If none of the additional three subjects experience a DLT (i.e., only 1 of 6 subjects in the cohort has a DLT), the dose will be escalated to the next highest level. If two or more of up to six subjects at a dose level have DLTs, enrollment to that cohort will stop and the dose will be considered to be above the maximum tolerated dose (MTD). The dose will then be decreased to the previous dose level or to a level intermediate to those previously evaluated. If only three subjects have been enrolled at this dose level, three additional subjects will be enrolled and treated to confirm that this is a tolerable dose level. The MTD will be the highest dose evaluated at which one of six (or less than ⅓^{rd} of subjects, if more than six subjects are enrolled in a cohort) have a DLT. A minimum of five DLT-evaluable subjects will be enrolled to any dose level being evaluated as the possible MTD.

The starting dose in Part A will be 0.25 mg/kg/day.

### Part B

Subjects in Phase I Part B will be treated with Compound 1 daily, three times per week every 3 weeks schedule (Schedule B, i.e., 3 days on, 18 days off. Cycle length is 3 weeks). Once at least two cohorts in Part A have been evaluated for safety through the DLT period, the first cohort in Part B may open for accrual. The initial dose in Part B will be determined based on the safety and tolerability of Part A subjects to that point. If the maximum grade of AEs related to Compound 1 in Part A is Grade 2, then the Dose Level 1 in Part B will be chosen so that the total dose in the new cohort in Part A and the initial cohort in Part B will deliver the same weekly total of Compound 1, as shown in Table 1. As an example, if the maximum grade AE in Part A Cohort 2 (e.g., 0.50 mg/kg) on the daily×5 schedule is Grade 2 and the SRC recommends a dose-escalation for Part A Cohort 3 (e.g., to 0.75 mg/kg: total dose over 5 days=3.75 mg/kg), the SRC could recommend Part B Cohort 1 open at a dose of up to 1.25 mg/kg/day such that the total over 3 days=3.75 mg/kg). If, however, the maximum grade AE in Part A Cohort 2 was >Grade 2, the daily dose of Part A Cohort 3 and Part B Cohort I would be identical (e.g., 0.75 mg/kg/day) (sec Table 2).

TABLE 1
Maximum AE in Part A ≤ Grade 2 Prior to Opening Part B

| Part A | | | | Part B | | |
|---|---|---|---|---|---|---|
| | | Total Dose | Maximum | | | Total Dose |
| Cohort | Dose | Over 5 Days | AE Grade | Cohort | Dose | Over 3 Days |
| A1 | 0.25 mg/kg/d | 1.25 mg/kg | Gr1 | | | |
| A2 | 0.50 mg/kg/d | 2.50 mg/kg | Gr2 | | | |
| A3 | 0.75 mg/kg/d | 3.75 mg/kg | *** | B1 | 1.25 mg/kg/d | 3.75 mg/kg |
| A4 | *** | *** | *** | B2 | *** | *** |

TABLE 2
Maximum AE in Part A > Grade 2 Prior to Opening Part B

| Part A | | | | Part B | | |
|---|---|---|---|---|---|---|
| | | Total Dose | Maximum | | | Total Dose |
| Cohort | Dose | Over 5 Days | AE Grade | Cohort | Dose | Over 3 Days |
| A1 | 0.25 mg/kg/d | 1.25 mg/kg | Gr1 | | | |
| A2 | 0.50 mg/kg/d | 2.50 mg/kg | Gr3 | | | |
| A3 | 0.75 mg/kg/d | 3.75 mg/kg | *** | B1 | 0.75 mg/kg/d | 2.25 mg/kg |
| A4 | *** | *** | *** | B2 | *** | *** |

Once Part B opens for accrual, dose levels in Part A and Part B will be based primarily on AEs and PK in each part.

Part B will follow a 3+3 design and follow the same dose level escalation/de-escalation rules as described in Part A. The DLT period for each subject in Phase I will be 3 weeks (i.e., 1 cycle). Subjects are considered evaluable for DLTs if in Part A, they receive at least four of the planned five daily doses in Cycle I and in Part B if they receive two of the planned three daily doses in Cycle 1 and are evaluable for safety through the 3-week DLT period or have had a DLT. Study subjects who are not evaluable for safety throughout the DLT period for reasons other than Compound 1 related toxicity will be replaced in the same dose cohort.

For Part A and Part B, after all subjects in a cohort have completed treatment through the DLT period or discontinued treatment due to a DLT, all available safety data will be reviewed, including DLTs and all available PK data for that cohort and make dose-level recommendations. While the primary basis for the dose level recommendations will be the occurrence of DLTs in a cohort, all available safety and PK data, including longer-term safety data from subjects treated in lower dose cohorts, will be considered.

A recommended Phase II dose (RP2D) will be determined for each schedule, as the MTD or a biologically active dose below the MTD. If an unequivocally biologically active (i.e., leading to confirmed responses) and tolerable dose is reached prior to the MTD, additional dose escalations may be stopped prior to defining an MTD. A minimum of six subjects will be evaluated at the RP2D in each of Part A and Part B.

Once the RP2D has been established in both Part A and Part B, Phase 2 expansion cohorts may open. One or both schedules (daily×3 every 3 weeks; daily×5 every 3 weeks) may be taken forward into Phase 2. One expansion cohort will enroll subjects with platinum-refractory epithelial ovarian cancer (including primary peritoneal and fallopian tube cancer) and one expansion cohort will enroll subjects with platinum-resistant SCLC. The protocol may be amended to include additional expansion cohorts based on emerging activity signals from Phase 1 or in tumor types in which topoisomerase 1 inhibitors have demonstrated efficacy.

The subject will receive treatment with Compound 1 and be monitored for safety and disease status by AE assessment, physical examination, laboratory tests, radiologic assessment, and ECG. PK and biomarker samples will be collected. All AEs and serious adverse events (SAEs) will be assessed using the National Cancer Institute Common Terminology Criteria for Adverse Events (NCI CTCAE) v5.0. Disease assessments by computed tomography (CT) or magnetic resonance imaging (MRI) will be conducted at protocol-specific intervals. Subjects may continue treatment as long as they are tolerating treatment without disease progression based on RECIST 1.1.

All study subjects will return to the study site approximately 30 days after their last dose of Compound 1 for an end-of-treatment (EOT) evaluation. Study subjects who have not had disease progression will continue to be followed until progression or start of a new systemic anti-cancer therapy. Additional follow-up will occur for ongoing Compound 1-related SAEs and clinically significant AEs until resolution or stabilization.

Approximately 112 subjects are planned for inclusion in this study, including approximately 30 subjects in Phase 1 and approximately 41 subjects in each of the Phase 2 expansion cohorts.

### Dose-Limiting Toxicity

DLTs will be assessed for all subjects enrolled in Phase I through their initial 3-week cycle of treatment (Cycle 1, the DLT period). Any AE occurring during the DLT period deemed at least possibly related to Compound 1 and meeting the criteria below will be designated a DLT. If intra-subject dose escalation for a subject is recommended and toxicity is observed upon escalation, this will not be considered a DLT, as it did not occur during the subject's initial 3-week cycle. This will, however, be considered in the SRC's subsequent dose-level recommendations.

Non-hematologic DLTs:
Any Grade 5 AE
Any Grade 4 AE other than electrolyte abnormalities that are asymptomatic and that resolve without clinical sequelae to ODGrade 2 within 2 days with or without medical management
≥Grade 3 aspartate aminotransferase (AST) and bilirubin >2× the upper limit of normal (ULN) (and >35% direct bilirubin) without evidence of biliary obstruction
Any other Grade 3 non-hematologic AE with the following exceptions:
   Grade 3 AST or alanine aminotransferase (ALT) that resolves to Grade 2 within 3 days
   Grade 3 nausea, vomiting, diarrhea, or mucositis that occurs without optimal prophylaxis or resolves with or without medical management to Grade 2 within 3 days
   Grade 3 rash that resolves with or without medical management to Grade 2 within 3 days
   Grade 3 fever that is uncomplicated and without clinical sequelae that resolves to Grade 2 within 3 days
   Grade 3 electrolyte abnormalities that are asymptomatic and without clinical sequelae that resolve with or without medical management to Grade 2 within 3 days
   Grade 3 fatigue or asthenia that resolves to Grade 2 within 7 days

### Hematologic DLTs

Any Grade 5 AE
Grade 3 or Grade 4 febrile neutropenia
Grade 4 neutropenia not associated with fever or clinically significant infection 7 days in duration or that is symptomatic
Grade 4 thrombocytopenia
Grade 3 thrombocytopenia associated with clinically significant bleeding

### Subject Inclusion Criteria

1. Subject has a histologically- or cytologically-diagnosed solid tumor which is advanced or metastatic and which has progressed on or following at least one systemic therapy regimen administered for advanced or metastatic disease or for which no approved therapy exists. Subject's prior treatment should include all approved regimens that have demonstrated a survival advantage for the subject's disease, stage, and line of therapy.

In Phase 1, preference will be given to subjects with the following tumor types:
Ovarian cancer
SCLC
Non-small cell lung cancer (NSCLC)
Hormone-receptor positive (HR+)/HER² negative (HER²−) breast cancer
Gastric cancer
Esophageal cancer
Colorectal cancer
Pancreas Cancer
Urothelial cancer
Sarcoma
Tumors with a BRCA1 or BRCA2 mutation

However, subjects with other solid tumors meeting eligibility are not excluded.

In Phase 2 expansion cohorts, subjects with epithelial ovarian cancer or SCLC must have disease progression or recurrence within 6 months of their last dose of platinum chemotherapy.
2. Age 18 years at the time of signing the informed consent form (ICF)
3. Has measurable disease per RECIST 1.1
4. An adequate tumor sample must be available from core needle biopsies or an incisional or excisional biopsy obtained during the Screening Period and following the subject's most recent systemic therapy. Previously obtained archival samples may be approved by the Medical Monitor for this inclusion criterion if the samples were taken following the most recent systemic therapy. The biopsy may not be from a previously irradiated lesion unless there has been disease progression in that lesion following the radiation therapy. The minimum adequate tumor sample is defined as the equivalent amount of tumor from approximately 3 core needle biopsies, with tissue from 4 to 5 cores being optimal.
5. Agrees to an on-treatment biopsy preferably of the same lesion from which the pre-Compound 1-treatment sample was obtained as long as the Investigator determines such biopsy can be performed with acceptable safety. A lesion from which 3 to 5 core biopsies can be obtained should be identified during the Screening Period.
6. Has provide written informed consent
7. ECOG Performance Status of 0 or 1
8. Adequate liver, renal, hematologic, pulmonary and coagulation function as follows
   a. Bilirubin ≤1.5× ULN
   b. AST (SGOT), ALT (SGPT) ≤3.0× ULN
   c. Serum creatinine ≤1.5× ULN and/or an estimated creatinine clearance of ≥60 ml/min based on the Cockcroft-Gault formula
   d. Absolute neutrophil count ≥1500/mm³
   e. Platelet count ≥100,000/mm³
   f. Hemoglobin ≥9 g/dL. Transfusion is permitted to meet this eligibility criterion
   g. International normalized ratio (INR) ≤1.5× ULN and activated partial thromboplastin time (aPTT) ≤1.5×p9 ULN. Study subjects on therapeutic doses of anticoagulation medication must have INR and/or aPTT≤the upper limit of the therapeutic range for intended use
   h. O₂ saturation >90% on room air
   i. Serum potassium, magnesium, and calcium or ionized calcium ≥LLN within 3 days of the first dose of Compound 1. Subjects may receive supplementation to meet this eligibility criterion.
9. A negative serum pregnancy test (for women of child-bearing potential) during Screening and a negative serum or urine pregnancy test on Cycle 1 Day 1 prior to the first dose of Compound 1
10. Women of child-bearing potential must agree to use highly effective contraceptive methods and avoid egg donation or preservation during the study treatment and for 4 months after the last dose of Compound 1. A woman is considered to be of child-bearing potential unless she:
   a. has had a hysterectomy, bilateral tubal occlusion, or bilateral oophorectomy:
   b, is age ≥60 years and is amenorrhoeic; or
   c, is age <60 years and has been amenorrhoeic for ≥12 months (including no irregular menses or spotting) in the absence of any medication which induces a menopausal state and has documented ovarian failure by serum estradiol and follicle-stimulating hormone levels within the institutional laboratory postmenopausal range).
11. Men of child-producing potential agree to use highly effective contraceptive methods and avoid sperm donation or preservation during the study treatment and for 4 months after the last dose of Compound 1. A man is considered to be of child-producing potential unless he has had a bilateral vasectomy with documented aspermia or a bilateral orchicctomy.

### Subject Exclusion Criteria

1. Cytotoxic chemotherapy, biologic agent, investigational agent, or radiation therapy
≤3 weeks prior to the first dose of Compound 1. The interval may be reduced to 2 weeks for bone-only radiation therapy or investigational agents not expected to be associated with AEs after 2 weeks of last administration, with Medical Monitor approval.
2. Small-molecule kinase inhibitors or hormonal agents ≤14 days prior to the first dose of Compound 1
3. Clinically significant AEs that have not returned to baseline or ≤Grade 1 based on NCI CTCAE v5.0
4. Subjects who are currently receiving any other anti-cancer or investigational agent(s)
5. Major surgery ≤4 weeks or minor surgery ≤14 days prior to the first dose of Compound 1. Adequate wound healing is required.
6. Clinically significant intercurrent disease including but not limited to:
   a. New York Heart Association Class III or IV heart failure
   b. Myocardial infarction or stroke ≤26 weeks prior to the first dose of Compound 1
   c. Unstable angina within ≤13 weeks prior to the first dose of Compound 1 unless the underlying disease has been corrected by procedural intervention (e.g. stent, bypass) and the subject has been angina-free for 4 weeks prior to the first dose of Compound 1
   d. Severe aortic stenosis
   e. Uncontrolled arrhythmia. Medical Monitor approval of subjects with any documented arrhythmia is required
   f. QTe>450 milliseconds by Fredericia criteria (QTcF) during Screening based on the average of 3 QTcF readings from 3 ECGs obtained 1 to 2 minutes apart and all within 10 minutes
   g. Clinically significant active infection requiring systemic antibiotic, antiviral or antifungal medication. Subjects must be medically stable, afebrile and not taking antimicrobial treatment for ≥3 days prior to the first dose of Compound 1 (excluding subjects with uncomplicated urinary tract or upper respiratory tract infections).
   h. Ongoing inflammatory diseases or process that could result in a low pH tissuc environment other than the tumor microenvironment unless approved by the Medical Monitor.
7. Subjects with primary CNS tumors or clinically active CNS metastases or carcinomatous meningitis. Brain metastases are considered stable if there is no progression when comparing brain MR¹ or CT scans obtained within 2 weeks prior to the planned first dose of Compound 1 to those obtained at least 4 weeks prior and the subject has not required doses of steroids equivalent to prednisone >10 mg daily, increasing doses of steroids, or other treatment to control brain metastases between the scans.
8. Subjects taking medications know to prolong the QT interval or associated with torsades de pointes unless the subject can safely discontinue these medications or change to comparable medications that do not significantly prolong the QT interval at least
5 half-lives or 7 days (whichever is longer) prior to the first dose of Compound 1
9. Subjects taking strong CYP3A4 or CYPIA2 inhibitors or inducers unless the subject can safely discontinue these medications or change to comparable medications that are not strong inducers or inhibitors of CYP 3A4 or CYPIA2 at least 5 half-lives prior to the first dose of Compound 1
10. Subjects taking sensitive substrates of CYP3A and CYP2B6 with a narrow therapeutic index unless the subject can safety discontinue these medications or change to comparable medications that are not sensitive substrates of CYP3A and CYP2B6 with a narrow therapeutic index
11. Known history of HIV infection
12. Active hepatitis B or C infection
13. A history of another malignancy, unless potentially curative treatment has been performed.
14. Pregnant of breastfeeding women.
15. Unwillingness or inability to comply with procedures required in this protocol.

### Subject Withdrawal Criteria

Subjects must discontinue treatment with Compound 1 for any of the following reasons:
Subject requests to stop study treatment.
Intolerable AEs which, in the opinion of the Investigator, indicate that continued study treatment is not in the best interest of the subject.
Intercurrent illness such that continued study treatment would put the subject at unacceptable risk or that impacts their ability to continue to comply with study requirements or freely provide informed consent.
Non-compliance with study drug treatment or protocol requirements eg, failure to attend study visits.
Use of unacceptable concomitant medication eg, non-protocol anti-cancer medications.
Female subjects who become pregnant or are breastfeeding.
Protocol-specified reasons for treatment discontinuation.
Sponsor termination of the study.

### Description of Study Drug and Administration

Compound 1 is aseptically manufactured as a lyophilized powder cake (target dose 20 mg/vial, with each vial containing 21.5 mg of active to account for overage) created from a sterile-filtered solution. The product is intended to be reconstituted with Water for Injection (WFI) (5.0 mL per vial used), then transferred to an infusion bag for dilution.

### Preliminary Results

Compound 1 was administered as a 1-hour IV infusion. As set forth above, the first patient on study was treated with a starting dose of 0.25 mg/kg, which was dosed daily five times a week every 3 weeks (Schedule A). Initially. an accelerated dose tritation was utilized, wherein single patient cohorts were accrued and the dose was escalated up to 100% for each new cohort until the occurrence of Grade 2 or greater treatment-related adverse events (TRAEs). A single patient was accrued to Cohort A1 (0.25 mg/kg). The dose was doubled to 0.5 mg/kg for Cohort A2, and 3 patients were accrued to this cohort due to the occurrence of greater than Grade 2 level adverse events. Findings from the initial four patients on Schedule A were:
The half-life of Compound 1 was about 20 hours, which was longer than the predicted half-life (about 8 hours), leading to significant accumulation with five consecutive days of dosing
Dosing on a mg/kg basis led to large variation in doses administered in a cohort (e.g., in one instance, one patient received more than twice the dose of another patient in the same cohort).

Notably, one patient with ovarian cancer had a partial response (PR) noted after Cycle 1. The patient's dose was reduced for Cycle 2 and restaging demonstrated a complete response (CR) and normalization of CA-125 biomarker levels.

As a result of the findings on Schedule A, patients were then enrolled on the following dosing schedules:

Schedule B: once daily three times per week every three weeks, on a mg/m² basis (in other words, the dosage was calculated based on the patient's body surface area)

Schedule C: once weekly on a mg/m² basis. Patients treated on this schedule received a sentinel dose on Day -14 followed by 2 weeks of no treatment to evaluate the full PK profile of Compound 1 and exatecan. Once weekly dosing started Cycle 1 Day 1.

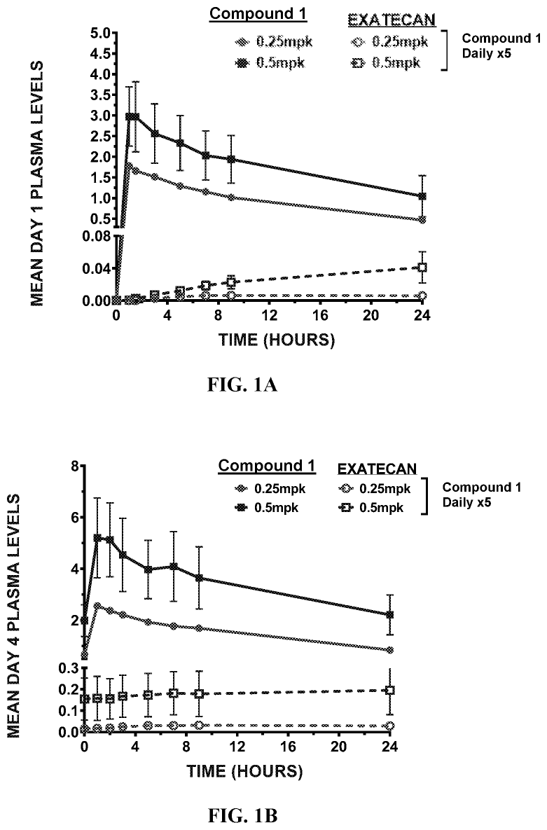

FIG. 1A shows the mean plasma levels of Compound 1 and exatecan (Cohort A) at 1 day

FIG. 1B shows the mean plasma levels of Compound 1 and exatecan (Cohort A) at 4 days.

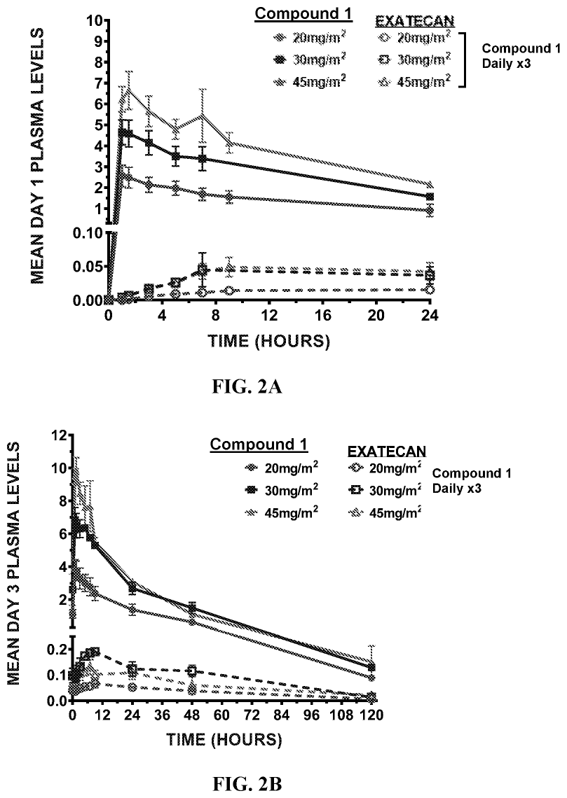

FIG. 2A shows the mean plasma levels of Compound 1 and exatecan (Cohort B) at 1 day.

FIG. 2B shows the mean plasma levels of Compound 1 and exatecan (Cohort B) at 3 days.

The following table summarizes the status of patients enrolled to Schedule B and Schedule C.

| | | | | | | |
|---|---|---|---|---|---|---|
| | | | Prior Treatment | Treatment- | | |
| Cohort/ | | Tumor | w/TOP1 | Related | Best | |
| Dose | Subject | Type | inhibitor | Adverse Events | Response | Status |

Schedule B: Daily × 3 Every 3 Weeks

| | | | | | | |
|---|---|---|---|---|---|---|
| B1 | 1-5 | Ovarian | Yes | Gr 1: Fatigue | PD | Off treatment after 2 |
| 20 | | | | Gr 2: | | cycles due to PD |
| mg/m² | | | | Dehydration | | |
| | 1-6 | Testicular | No | No TRAEs | SD | Off treatment after 2 |
| | | | | reported | (10% | cycles due to |
| | | | | | increase) | patient/physician |
| | | | | | | decision |
| | 1-7 | Esophageal/ | No | Gr 1: Fatigue, | NE | Off treatment due to |
| | | GEJ | | urinary | | SAE - Bile duct stenosis |
| | | | | frequency | | and sepsis not related to |
| | | | | Gr 2: Anemia | | Compound 1. |
| | | | | (baseline Hgb | | |
| | | | | 9.4) | | |
| B2 | 2-4 | Appendiceal | Yes | Gr 1: Anemia | SD | Off treatment after Cycle |
| 30 | | | | Gr 2: Nausea, | (4% | 2. SAE of small bowel |
| mg/m² | | | | vomiting | decrease) | obstruction due to |
| | | | | | | disease. Went to hospice. |
| | 2-5 | Breast | Yes | Gr 3: | SD | Off treatment after 5 |
| | | | | Neutropenia | (9.6% | cycles due to clinical |
| | | | | (C4D11) | increase) | progression |
| | 2-6 | Breast | No | Gr 1: Fatigue, | SD | On treatment: Cycle 6 |
| | | | | nausea | (29.1% | |
| | | | | Gr 2: | decrease) | |
| | | | | Neutropenia, | | |
| | | | | WBC decrease | | |
| B3 | 2-8 | Osteosarcoma | Unknown | No TRAEs | NE | Off treatment. Large |
| 45 | | | | reported | | pericardial effusion |
| mg/m² | | | | | | noted on pretreatment |
| | | | | | | CT scan. ECHO done |
| | | | | | | C1D2 demonstrating |
| | | | | | | cardiac compromise. |
| | | | | | | Had pericardial window |
| | | | | | | on C1D2 and removed |
| | | | | | | from study. |
| | 1-9 | Colorectal | Yes | Gr 1: Vomiting, | SD | Off treatment in Cycle 4 |
| | | | | diarrhea | (16% | due to PD |
| | | | | Gr 2: Nausea | decrease) | |
| | | | | Gr3: AST/ALT | | |
| | | | | elevation | | |
| | | | | (resolved to Gr 1 | | |
| | | | | in 2-3 days), | | |
| | | | | anemia | | |
| | 3-1 | Colorectal | Yes | Gr 1: Headache | SD | On treatment: Cycle 3 |
| | | | | Gr 2: Nausea, | | |
| | | | | WBC decrease, | | |
| | | | | ANC decrease, | | |
| | | | | diarrhea | | |
| | 2-11 | Ovarian | No | Gr 1: Nausea, | SD | On treatment: Cycle 3; |
| | | | | lipase increase, | | |
| | | | | alopecia, AST | | |
| | | | | elevation | | |
| | | | | Gr 2: Anemia | | |
| | | | | (C1D1 Hgb = 10.3) | | |
| | | | | Gr 3 | | |
| | | | | Neutropenia | | |
| | | | | (resolved in 2 | | |
| | | | | days) | | |
| B4 | 5-2 | Colorectal | Yes | Gr2: Vomiting, | NE | Off treatment in Cycle 1; |
| 60 | | | | dehydration | | patient and family chose |
| mg/m² | | | | Gr 3: Febrile | | hospice care |
| | | | | neutropenia, | | |
| | | | | anemia | | |
| | | | | Gr 4: | | |
| | | | | Neutropenia, | | |
| | | | | thrombocytopenia | | |
| | 2-12 | Breast | No | Gr 1: Vomiting | Pending | On treatment in Cycle 1 |
| | | | | Gr 2: Colitis | | pending decision about |
| | | | | Gr3: Febrile | | continuing on treatment. |
| | | | | neutropenia | | |
| | | | | Gr 4: | | |
| | | | | Neutropenia, | | |
| | | | | thrombocytopenia | | |

Schedule C: Once Weekly

| | | | | | | |
|---|---|---|---|---|---|---|
| C1 | 2-1 | Pancreas | Yes | No TRAES | PD | Off treatment in Cycle 2 |
| 20 | | | | reported | | due to PD |
| mg/m² | 2-2 | Colon | Yes | No TRAEs | NE | Off treatment after PK |
| | | | | reported | | period. SAE: Death due |
| | | | | | | to pneumonia. Not |
| | | | | | | evaluable for DLTs. |
| | | | | | | Subject replaced in |
| | | | | | | cohort. |
| | 2-3 | Ovarian | No | Gr 2: Fatigue | NE | Off treatment. SAEs of |
| | | | | | | dyspnea and partial |
| | | | | | | small bowel obstruction |
| | | | | | | due to disease. Not |
| | | | | | | evaluable for DLTs. |
| | | | | | | Subject replaced in |
| | | | | | | cohort |
| | 1-8 | Pancreas | Yes | Gr 1: Vomiting | SD | On treatment: Cycle 3 |
| | | | | Gr 2: Nausea | | |
| | 2-9 | Breast | No | No TRAEs | PR | On treatment: Cycle 3 |
| | | | | reported | (39.7% | |
| | | | | | decrease) | |

CR = Complete Response;
SD = Stable Disease;
PR = Partial Response;
PD = Progressive Disease;
PE = Pending Evaluation;
NE = Not Evaluable

As shown in the above table, several patients enrolled in the study derived clinical benefit. For example:

Patient 2-5 (Cohort B2) was a breast cancer patient with multiple lines of prior therapy including the topoisomerase inhibitor trastuzumab deruxtecan from 28 months before disease progression on this therapy. After 4 cycles on Compound 1 the patient demonstrated stable diseasc.

Patient 2-6 (Cohort B2) was a breast cancer patient having a large chest wall lesion, causing pain. Two cycles of Compound 1 demonstrated a 20% decrease (from pretreatment) in the chest wall lesion, resolution of pain, and decrease in numerous non-target lesions. Four cycles of Compound 1 demonstrated a further reduction of both the target lesions (decrease of 29.1% from pretreatment) and further decrease in non-target lesions.

Patient 1-9 (Cohort B3) was a colorectal cancer patient with prior therapy including the irinotecan-containing regiment of FOLFIRI+bevacizumab. The investigator reported a 16% reduction in target lesions after two cycles of Compound 1 treatment and resolution of bowel symptoms.

Patient 3-1 (Cohort B3) was a colorectal cancer patient with prior therapy including two lines of irinotecan-containing regimens (FOLFUR¹+bevacizumab; FOLFIRINOX+bevacizumab). Two cycles of Compound 1 demonstrated stable disease with decreasing carcinoembryonic antigen (CEA).

Patient 2-11 (Cohort B3) was an ovarian cancer patient with multiple lines of prior therapy. Two cycles of Compound 1 demonstrated stable disease with decreasing CA-125 biomarker levels.

Patient 2-9 (Cohort C1) was a breast cancer patient with multiple lines of prior therapy. Two cycles of Compound 1 demonstrated a partial response.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

What is claimed is:

1. A method of treating cancer in a patient, comprising administering to said patient a compound of Formula (I): 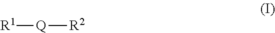
or a pharmaceutically acceptable salt thereof, wherein:
R¹ is a peptide;
R² is a small molecule topoisomerase I targeting moiety, which binds to topoisomerase I; and
Q is a linker, which is covalently linked to moiety R¹ and R²;
wherein the compound, or the pharmaceutically acceptable salt thereof, is administered in a daily dose of about 0.1 mg/kg to about 1.5 mg/kg as measured by the amount of the free form of the compound.

2. The method of claim 1, wherein R¹ is a conformationally restricted peptide.

3. The method of claim 1, or a pharmaceutically acceptable salt thereof, wherein R¹ is a peptide capable of selectively delivering -QR² across a cell membrane having an acidic or hypoxic mantle having a pH less than about 6.0.

4. The method of claim 1, or a pharmaceutically acceptable salt thereof, wherein R² is a radical of camptothecin, opotecan, irinotecan (CPT-11), silatecan (DB-67, AR-67), cositecan (BNP-1350), lurtotecan, gimatecan (ST1481), belotecan (CKD-602), rubitecan, topotecan, deruxtecan, or exatecan.

5. A method of treating cancer in a patient, comprising administering to said patient Compound 1 having the structure: 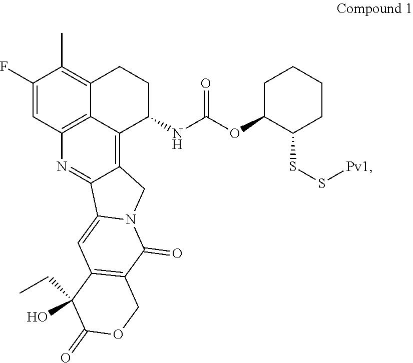
or a pharmaceutically acceptable salt thereof, wherein Pv1 is a peptide comprising the following sequence:
ADDQNPWRAYLDLLFPTDTLLLDLLWCG (SEQ ID NO: 1); and
wherein Compound 1 or the pharmaceutically acceptable salt thereof is administered in a daily dose of about 0.1 mg/kg to about 1.5 mg/kg as measured by the amount of the free form of Compound 1.

6. The method of claim 5, wherein Compound 1, or a pharmaceutically acceptable salt thereof, is administered to said patient intravenously.

7. The method of claim 5 or 6, wherein Compound 1, or a pharmaceutically acceptable salt thereof, is administered in a daily dose of about 0.25 mg/kg to about 1.5 mg/kg as measured by the amount of the free form of Compound 1.

8. The method of claim 5 or 6, wherein Compound 1, or a pharmaceutically acceptable salt thereof, is administered in a daily dose of about 0.25 mg/kg to about 1.25 mg/kg as measured by the amount of the free form of Compound 1.

9. The method of claim 5 or 6, wherein Compound 1, or a pharmaceutically acceptable salt thereof, is administered in a daily dose of about 0.25 mg/kg to about 0.75 mg/kg as measured by the amount of the free form of Compound 1.

10. The method of claim 5 or 6, wherein Compound 1, or a pharmaceutically acceptable salt thereof, is administered in a daily dose of about 0.25 mg/kg as measured by the amount of the free form of Compound 1.

11. The method of claim 5 or 6, wherein Compound 1, or a pharmaceutically acceptable salt thereof, is administered in a daily dose of about 0.50 mg/kg as measured by the amount of the free form of Compound 1.

12. The method of claim 5 or 6, wherein Compound 1, or a pharmaceutically acceptable salt thereof, is administered in a daily dose of about 0.75 mg/kg as measured by the amount of the free form of Compound 1.

13. The method of claim 5 or 6, wherein Compound 1, or a pharmaceutically acceptable salt thereof, is administered in a daily dose of about 1.25 mg/kg as measured by the amount of the free form of Compound 1.

14. The method of any one of claims 5-13, wherein Compound 1, or a pharmaceutically acceptable salt thereof, is administered in a continuous dosing schedule.

15. The method of any one of claims 5-13, wherein Compound 1, or a pharmaceutically acceptable salt thereof, is administered in an intermittent dosing schedule comprising one or more cycles, wherein each cycle comprises a first period of consecutive days wherein Compound 1, or the pharmaceutically acceptable salt thereof, is administered and a second period of consecutive days wherein Compound 1, or the pharmaceutically acceptable salt thereof, is not administered.

16. The method of claim 15, wherein the total length of each cycle is 7 days to 60 days.

17. The method of claim 15, wherein the total length of each cycle is 14 days to 30 days.

18. The method of claim 15, wherein the total length of each cycle is 21 days.

19. The method of any one of claims 15-18, wherein the first period is 5 days and the second period is the remainder of the cycle.

20. The method of any one of claims 15-18, wherein the first period is 3 days and the second period is the remainder of the cycle.

21. The method of any one of claims 15-18, wherein the first period is 2 days and the second period is the remainder of the cycle.

22. The method of any one of claims 15-18, wherein the first period is 1 day and the second period is the remainder of the cycle.

23. A method of treating cancer in a patient, comprising administering to said patient a compound of Formula (1): 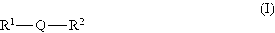
or a pharmaceutically acceptable salt thereof, wherein:
R¹ is a peptide;
R¹ is a small molecule topoisomerase I targeting moiety, which binds to topoisomerase I; and
Q is a linker, which is covalently linked to moicty R¹ and R²;
wherein the compound or the pharmaceutically acceptable salt thereof is administered in a daily dose of about 5 mg/m² to about 100 mg/m² as measured by the amount of the free form of the compound.

24. The method of claim 23, wherein R¹ is a conformationally restricted peptide.

25. The method of claim 23, or a pharmaceutically acceptable salt thereof, wherein R¹ is a peptide capable of selectively delivering -QR² across a cell membrane having an acidic or hypoxic mantle having a pH less than about 6.0.

26. The method of claim 23, or a pharmaceutically acceptable salt thereof, wherein R² is a radical of camptothecin, opotecan, irinotecan (CPT-11), silatecan (DB-67, AR-67), cositecan (BNP-1350), lurtotecan, gimatecan (ST1481), belotecan (CKD-602), rubitecan, topotecan, deruxtecan, or exatecan.

27. A method of treating cancer in a patient, comprising administering to said patient Compound 1 having the structure: 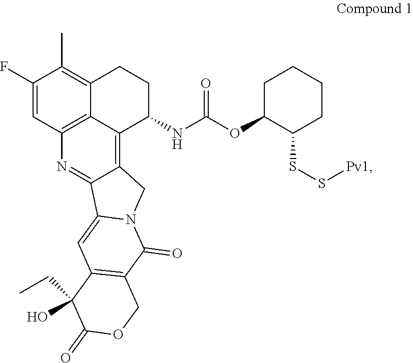
or a pharmaceutically acceptable salt thereof, wherein Pv1 is a peptide comprising the following sequence:
ADDQNPWRAYLDLLFPTDTLLLDLLWCG (SEQ ID NO: 1); and
wherein Compound 1 or the pharmaceutically acceptable salt thereof is administered in a daily dose of about 5 mg/m² to about 100 mg/m² as measured by the amount of the free form of Compound 1.

28. The method of claim 27, wherein Compound 1, or a pharmaceutically acceptable salt thereof, is administered to said patient intravenously.

29. The method of claim 27 or 28, wherein Compound 1, or a pharmaceutically acceptable salt thereof, is administered in a daily dose of about 10 mg/m² to about 80 mg/m² as measured by the amount of the free form of Compound 1.

30. The method of claim 27 or 28, wherein Compound 1, or a pharmaceutically acceptable salt thereof, is administered in a daily dose of about 20 mg/m² to about 60 mg/m² as measured by the amount of the free form of Compound 1.

31. The method of claim 27 or 28, wherein Compound 1, or a pharmaceutically acceptable salt thereof, is administered in a daily dose of about 20 mg/m² to about 45 mg/m² as measured by the amount of the free form of Compound 1.

32. The method of claim 27 or 28, wherein Compound 1, or a pharmaceutically acceptable salt thereof, is administered in a daily dose of about 20 mg/m² as measured by the amount of the free form of Compound 1.

33. The method of claim 27 or 28, wherein Compound 1, or a pharmaceutically acceptable salt thereof, is administered in a daily dose of about 30 mg/m² as measured by the amount of the free form of Compound 1.

34. The method of claim 27 or 28, wherein Compound 1, or a pharmaceutically acceptable salt thereof, is administered in a daily dose of about 45 mg/m² as measured by the amount of the free form of Compound 1.

35. The method of claim 27 or 28, wherein Compound 1, or a pharmaceutically acceptable salt thereof, is administered in a daily dose of about 60 mg/m² as measured by the amount of the free form of Compound 1.

36. The method of any one of claims 27-35, wherein Compound 1, or a pharmaceutically acceptable salt thereof, is administered in a continuous dosing schedule.

37. The method of any one of claims 27-36, wherein Compound 1, or a pharmaceutically acceptable salt thereof, is administered once weekly.

38. The method of any one of claims 27-35, wherein Compound 1, or a pharmaceutically acceptable salt thereof, is administered in an intermittent dosing schedule comprising one or more cycles, wherein each cycle comprises a first period of consecutive days wherein Compound 1, or the pharmaceutically acceptable salt thereof, is administered and a second period of consecutive days wherein Compound 1, or the pharmaceutically acceptable salt thereof, is not administered.

39. The method of claim 38, wherein the total length of each cycle is 7 days to 60 days.

40. The method of claim 38, wherein the total length of cach cycle is 14 days to 30 days.

41. The method of claim 38, wherein the total length of each cycle is 21 days.

42. The method of any one of claims 38-41, wherein the first period is 5 days and the second period is the remainder of the cycle.

43. The method of any one of claims 38-41, wherein the first period is 3 days and the second period is the remainder of the cycle.

44. The method of any one of claims 38-41, wherein the first period is 2 days and the second period is the remainder of the cycle.

45. The method of any one of claims 38-41, wherein the first period is 1 day and the second period is the remainder of the cycle.

46. The method of any one of claims 1-45, wherein the cancer is selected from ovarian cancer, small cell lung cancer (SCLC), non-small cell lung cancer (NSCLC), breast cancer, gastric cancer, esophageal cancer, colorectal cancer, pancreatic cancer, urothelial cancer, and sarcoma.

47. The method of any one of claims 1-46 wherein the patient has failed at least one previous treatment for cancer.

48. The method of any one of claims 1-47, wherein the method further comprises administering Compound 1, or a pharmaceutically acceptable salt thereof, in combination with an additional therapy.

49. A method of administering Compound 1, or a pharmaceutically acceptable salt thereof, to a patient in need of treatment, comprising parenterally delivering to said patient Compound 1 having the structure: 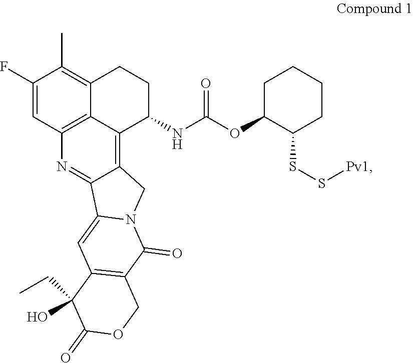
or a pharmaceutically acceptable salt thereof, wherein Pv1 is a peptide comprising the following sequence:
ADDQNPWRAYLDLLFPTDTLLLDLLWCG (SEQ ID NO: 1); and
wherein Compound 1 or the pharmaceutically acceptable salt thereof is administered in a daily dose of about 0.1 mg/kg to about 1.5 mg/kg as measured by the amount of the free form of Compound 1.

50. The method of claim 49, wherein Compound 1, or a pharmaceutically acceptable salt thereof, is administered to said patient intravenously.

51. The method of claim 49 or 50, wherein Compound 1, or a pharmaceutically acceptable salt thereof, is administered in a daily dose of about 0.25 mg/kg to about 1.5 mg/kg as measured by the amount of the free form of Compound 1.

52. The method of claim 49 or 50, wherein Compound 1, or a pharmaceutically acceptable salt thereof, is administered in a daily dose of about 0.25 mg/kg to about 1.25 mg/kg as measured by the amount of the free form of Compound 1.

53. The method of claim 49 or 50, wherein Compound 1, or a pharmaceutically acceptable salt thereof, is administered in a daily dose of about 0.25 mg/kg to about 0.75 mg/kg as measured by the amount of the free form of Compound 1.

54. The method of claim 49 or 50, wherein Compound 1, or a pharmaceutically acceptable salt thereof, is administered in a daily dose of about 0.25 mg/kg as measured by the amount of the free form of Compound 1.

55. The method of claim 49 or 50, wherein Compound 1, or a pharmaceutically acceptable salt thereof, is administered in a daily dose of about 0.50 mg/kg as measured by the amount of the free form of Compound 1.

56. The method of claim 49 or 50, wherein Compound 1, or a pharmaceutically acceptable salt thereof, is administered in a daily dose of about 0.75 mg/kg as measured by the amount of the free form of Compound 1.

57. The method of claim 49 or 50, wherein Compound 1, or a pharmaceutically acceptable salt thereof, is administered in a daily dose of about 1.25 mg/kg as measured by the amount of the free form of Compound 1.

58. The method of any one of claims 49-57, wherein Compound 1, or a pharmaceutically acceptable salt thereof, is administered in a continuous dosing schedule.

59. The method of any one of claims 49-57, wherein Compound 1, or a pharmaceutically acceptable salt thereof, is administered in an intermittent dosing schedule comprising one or more cycles, wherein cach cycle comprises a first period of consecutive days wherein Compound 1, or the pharmaceutically acceptable salt thereof, is administered and a second period of consecutive days wherein Compound 1, or the pharmaceutically acceptable salt thereof, is not administered.

60. The method of claim 59, wherein the total length of each cycle is 7 days to 60 days.

61. The method of claim 59, wherein the total length of each cycle is 14 days to 30 days.

62. The method of claim 59, wherein the total length of each cycle is 21 days.

63. The method of any one of claims 59-62, wherein the first period is 5 days and the second period is the remainder of the cycle.

64. The method of any one of claims 59-62, wherein the first period is 3 days and the second period is the remainder of the cycle.

65. The method of any one of claims 59-62, wherein the first period is 2 days and the second period is the remainder of the cycle.

66. The method of any one of claims 59-62, wherein the first period is 1 day and the second period is the remainder of the cycle.

67. The method of any one of claims 49-66, wherein the method further comprises administering Compound 1, or a pharmaceutically acceptable salt thereof, in combination with an additional therapy.

68. A method of administering Compound 1, or a pharmaceutically acceptable salt thereof, to a patient in need of treatment, comprising parenterally delivering to said patient Compound 1 having the structure: 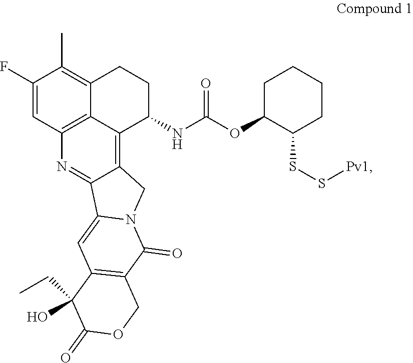
or a pharmaceutically acceptable salt thereof, wherein Pv1 is a peptide comprising the following sequence:
ADDQNPWRAYLDLLFPTDTLLLDLLWCG (SEQ ID NO: 1); and
wherein Compound 1 or the pharmaceutically acceptable salt thereof is administered in a daily dose of about 5 mg/m² to about 100 mg/m² as measured by the amount of the free form of Compound 1.

69. The method of claim 68, wherein Compound 1, or a pharmaceutically acceptable salt thereof, is administered to said patient intravenously.

70. The method of claim 68 or 69, wherein Compound 1, or a pharmaceutically acceptable salt thereof, is administered in a daily dose of about 10 mg/m² to about 80 mg/m² as measured by the amount of the free form of Compound 1.

71. The method of claim 68 or 69, wherein Compound 1, or a pharmaceutically acceptable salt thereof, is administered in a daily dose of about 20 mg/m² to about 60 mg/m² as measured by the amount of the free form of Compound 1.

72. The method of claim 68 or 69, wherein Compound 1, or a pharmaceutically acceptable salt thereof, is administered in a daily dose of about 20 mg/m² to about 45 mg/m² as measured by the amount of the free form of Compound 1.

73. The method of claim 68 or 69, wherein Compound 1, or a pharmaceutically acceptable salt thereof, is administered in a daily dose of about 20 mg/m² as measured by the amount of the free form of Compound 1.

74. The method of claim 68 or 69, wherein Compound 1, or a pharmaceutically acceptable salt thereof, is administered in a daily dose of about 30 mg/m² as measured by the amount of the free form of Compound 1.

75. The method of claim 68 or 69, wherein Compound 1, or a pharmaceutically acceptable salt thereof, is administered in a daily dose of about 45 mg/m² as measured by the amount of the free form of Compound 1.

76. The method of claim 68 or 69, wherein Compound 1, or a pharmaceutically acceptable salt thereof, is administered in a daily dose of about 60 mg/m² as measured by the amount of the free form of Compound 1.

77. The method of any one of claims 68-76, wherein Compound 1, or a pharmaceutically acceptable salt thereof, is administered in a continuous dosing schedule.

78. The method of any one of claims 68-77, wherein Compound 1, or a pharmaceutically acceptable salt thereof, is administered once weekly.

79. The method of any one of claims 68-76, wherein Compound 1, or a pharmaceutically acceptable salt thereof, is administered in an intermittent dosing schedule comprising one or more cycles, wherein each cycle comprises a first period of consecutive days wherein Compound 1, or the pharmaceutically acceptable salt thereof, is administered and a second period of consecutive days wherein Compound 1, or the pharmaceutically acceptable salt thereof, is not administered.

80. The method of claim 79, wherein the total length of each cycle is 7 days to 60 days.

81. The method of claim 79, wherein the total length of each cycle is 14 days to 30 days.

82. The method of claim 79, wherein the total length of each cycle is 21 days.

83. The method of any one of claims 79-82, wherein the first period is 5 days and the second period is the remainder of the cycle.

84. The method of any one of claims 79-82, wherein the first period is 3 days and the second period is the remainder of the cycle.

85. The method of any one of claims 79-82, wherein the first period is 2 days and the second period is the remainder of the cycle.

86. The method of any one of claims 79-82 wherein the first period is 1 day and the second period is the remainder of the cycle.


SEQUENCE LISTING (5 sequences)

SEQ ID NO: 1 - protein, 28 aa, Artificial
  synthetic peptide
ADDQNPWRAYLDLLFPTDTLLLDLLWCG

SEQ ID NO: 2 - protein, 35 aa, Artificial
  synthetic peptide
AEQNPIYWARYADWLFTTPLLLLDLALLVDADECG

SEQ ID NO: 3 - protein, 32 aa, Artificial
  synthetic peptide
ADDQNPWRAYLDLLFPTDTLLLDLLWDADECG

SEQ ID NO: 4 - protein, 39 aa, Artificial
  synthetic peptide
AAEQNPIYWARYADWLFTTPLLLLDLALLVDADEGTKCG

SEQ ID NO: 5 - protein, 37 aa, Artificial
  synthetic peptide
AAEQNPIYWARYADWLFTTPLLLLDLALLVDADEGTC